(19) United States
(12) Patent Application Publication
(10) Pub. No.: US 2016/0361078 A1
(43) Pub. Date: Dec. 15, 2016

(54) ORAL DEBRIS EXTRACTOR

(71) Applicants: Vincent Dario Pagliacci, Grimsby (CA); Linda Marie Pagliacci, Grimsby (CA)

(72) Inventors: Vincent Dario Pagliacci, Grimsby (CA); Linda Marie Pagliacci, Grimsby (CA)

(21) Appl. No.: 15/122,351

(22) PCT Filed: Feb. 27, 2015
(86) PCT No.: PCT/CA2015/000124

§ 371 Date: Aug. 29, 2016

Related U.S. Application Data
(60) Provisional application No. 61/966,647, filed on Feb. 27, 2014.
(60) Provisional application No. 61/952,355, filed on Mar. 13, 2014.
(60) Provisional application No. 62/062,685, filed on Oct. 10, 2014.

(51) Int. Cl. A61B 17/24; A61M 1/00; A61C 17/02

(52) U.S. Cl. CPC A61B 17/24; A61C 17/02; A61M 1/0072; A61M 1/008; A61B 2217/005; A61M 2205/075; A61M 2210/0625

(57) ABSTRACT

This invention relates to oral debris extractors and devices for the removal of oral debris from an oral cavity of humans or mammals. Oral debris extractors include tonsil stone extractors for removal of tonsilloliths and tonsil stones in an oral cavity as well as extractors for removal of other types of oral debris present in an oral cavity. The invention relates to uses of oral debris extractor devices to remove all types of oral debris from the oral cavity of humans or mammals as well as for use to explore and assess the presence of oral debris and tonsil stones in the oral cavity and in the tissues of the oral cavity. Methods of using an oral debris extractor device to explore the oral cavity and to remove oral debris are also described. The oral debris extractor is hand-held and does not require any external power source or battery to be operated. A curved shaft provides ergonomic and other advantages. The oral debris extractor can be manufactured using recyclable materials. In some embodiments the device is suitable for disposable after a single use.

### REFERENCE TO RELATED APPLICATIONS

This application claims priority to U.S. Provisional Application No. 61/966,647 filed Feb. 27, 2014; U.S. Provisional Application No. 61/952,355 filed Mar. 13, 2014; and U.S. Provisional Application No. 62/062,685 filed Oct. 10, 2014; all of which are specifically and entirely incorporated by reference.

### FIELD

This invention relates to oral debris extractors. Devices of the present invention relate to the removal of oral debris from an oral cavity of a human or a mammal. Oral debris extractors of the present invention include, for example, tonsil stone extractors and related embodiments for removal of tonsilloliths and tonsil stones in an oral cavity. The invention also relates to devices, and their uses, to remove all types of oral debris from an oral cavity of a human or a mammal. The invention further relates to methods of using an oral debris extractor device to explore the oral cavity and to remove oral debris from an oral cavity of a human or a mammal. The invention further relates to a hand-held oral debris extractor which does not require any external power source or battery to be operated. The invention further relates to an oral debris extractor which can be manufactured in recyclable materials and used in humans and mammals in the oral cavity.

### BACKGROUND

Oral debris can accumulate in the oral cavity of humans and other mammals. Oral debris is any debris that has accumulated in the oral cavity, including the mouth and the throat. Oral debris is comprised of foreign material, food deposits or loose food particles including, without limitation, bacteria, dead cells, food particles and mucous. When oral debris is not completely eradicated during regular oral hygiene and thereby accumulates in the oral cavity, even in minute amounts, the accumulations can lead to discomfort, poor oral health and the development of consequential conditions of poor health eventually causing deterioration of the health of the oral cavity.

Present in the oral cavity are lymphatic oral tissues and organ systems. The oral cavity includes tonsils and other glands in which oral debris may accumulate and it is not readily or easily possible to remove the debris from these tissues particularly when the debris becomes embedded within the tissue. Over time, as the debris accumulates it can harden through a process called calcification causing small stones or other hardened pieces of debris which eventually may become painful. The debris can cause halitosis because of the presence of anaerobic bacteria which have accumulated in the tissues and is found in the stones or debris which have calcified in the oral cavity. Debris can lead to other conditions as well including infections and inflammation of the oral cavity. A patient or individual may experience difficulty swallowing, or a feeling like something is caught in their throat, or a feeling of a sore throat, or post nasal drip and even ear pain.

Tonsil stones, also referred to as tonsilloliths, may continue to grow as oral debris continues to accumulate in the oral cavity, if they are not removed. Larger stones are more discomforting and in some instances become painful if not removed. In patients and individuals who have other health conditions such as neurologic conditions or have experienced a stroke, it may not be possible for them to optimize their oral health simply by normal dental oral hygiene. In these instances it is important to have an option to assist the patient or individual to explore the oral cavity for oral debris accumulations and remove oral debris from the oral cavity.

Tonsils are a pair of soft tissue masses located at the rear of the pharynx. Each tonsil is composed of tissue similar to lymph nodes, covered by pink mucosa. Running through the mucosa of each tonsil are pits, folds, crypts, other indentations, crevices and spaces often referred to as crypts, in which oral debris may accumulate. Humans have tonsils as do some other mammals including, for example, dogs. Due to large numbers of people who still have their tonsils, the numbers of adults with tonsil stones is rising. Tonsil stones may also occur in children.

When debris accumulates in the crypts of one or more tonsils, it can turn into a solid mass, aided by calcification and the presence of anaerobic bacteria, and cause irritation. Tonsillioths, tonsillar calculi, or tonsil stones, as they are more commonly referred to, are clusters of such calcified materials and hardened pieces of debris that are found in the crypts and pits of the tonsils. Bacteria, dead cells and mucous can become trapped and, over time, hardened, embedding themselves inside the crypts and pits of the tonsils. They are also known to form in the throat and on the roof of the mouth and in association with other glands wherever there are places in the mouth that the debris can hide and become lodged or embedded. The stones may be partially or completely hidden in the folds or in the crypts. Even if not visible with the eye the stones can be felt by the patient or individual.

Tonsil stones harbor bacteria and when they reside on the tonsil this can lead to inflammation and/or irritation of the region. This region of the body also has connectivity to the ear and nose. Underlying health conditions of a patient may make the person more prone to tonsil stones. Overall, tonsil stones can also be associated with a number of related health issues as well as social issues. According to some physicians, certain prescription medications which cause dry mouth can contribute to the problem of tonsil stones by increasing the activity of anaerobic bacteria.

Many neurologic illnesses and conditions create situations which make an individual more prone to the accumulation of oral debris in the oral cavity and throat region. Strokes, ALS, and Multiple Sclerosis are but a few examples of these types of conditions. These neurologic conditions, and others, may also result in dysphagia in the individual or patient which in and of itself, can leave the individual prone to increased difficulties clearing the oral cavity, and swallowing, thereby leading to increased oral debris in the oral cavity and throat.

Dysphagia is defined by the Mayo Clinic as difficulty swallowing. Simply put, this means it takes more time and effort to move food or liquid from the mouth to the stomach. This condition may also be associated with pain. In some cases, individuals are unable to swallow completely, or have difficulty swallowing liquids or solids safely. In some instances it is difficult or impossible to even swallow saliva. As noted by the Mayo Clinic at their website http://www.mayoclinic.org/diseases-conditions/dysphagia/basics/definition/con-20033444 the causes of swallowing problems vary and all of these diseases and conditions can contribute to difficulties clearing the oral cavity of debris.

Accordingly, while dysphagia may result from neurologic conditions it can also result from other conditions as well, including cancers, muscular disorders, infections and physical obstructions from a number of causes as would be known to persons skilled in the art. Regardless of the underlying cause of the dysphagia, the dysphagia itself may lead to increased accumulation of oral debris in the oral cavity because of the difficulty in swallowing. On the other hand, it is also the case that increased oral debris may lead to dysphagia. The presence of tonsil stones, for example, may lead to pain or difficulties with swallowing. Pain when swallowing is also known as odynophagia.

As noted by the Royal College of Speech and Language Therapists, the process of swallowing is something that the average individual will do about 600 times per day. It is a complex physical action involving about 50 muscles and many nerves. The nerves involved in swallowing connect the pharynx and the esophagus to the swallowing center in the brain. It is known by those in the field that the process of swallowing actually involves four phases:
1. Oral preparatory phase
2. Oral transit phase
3. Pharyngeal phase
4. Esophageal phase.

The details of these phases and how they work in the human body are understood by persons trained in these fields. Consider, for example, the Royal College of Speech and Language Therapists as a reliable source of documentation on dysphagia as are many others that would be known to persons skilled in the art, including the National Institutes of Health (NIH). Consider, for example, the NIH discussions of dysphagia at this hyperlink http://www.nided.nih.gov/health/voice/pages/dysph.aspx. Dysphagia is related to numerous conditions and illnesses creating oral health and hygiene concerns as oral debris accumulates due to swallowing difficulties.

It is considered that oropharyngeal dysphagia has high morbidity, mortality, and cost in health in the population at large. Estimates of the prevalence of dysphagia among individuals older than 50 years range from 16% to 22%. Within health care institutions, it is estimated that 12% to 13% of patients in short-term care hospitals and up to 60% of nursing home occupants have feeding difficulties of which a substantial proportion are troubled by oral or oropharyngeal dysphagia (as opposed to esophageal dysphagia). Specific populations of patients are estimated to have an increased incidence of dysphagia such as those with head injuries, cerebrovascular accidents, or Parkinson's disease. In these populations it is estimated that there is a 20% to 40% prevalence of oropharyngeal dysphagia.

Market assessment reflects a need for an oral debris extractor in accordance with the present invention across the entire population with at least 1 in 4 persons reflecting a need in the age group under 75 years of age and 1 in 2 persons reflecting a need in the age group over 75 years old.

Some of the health and social issues which occur in association with tonsil stones which are produced as a result of an accumulation of oral debris in the oral cavity include:
(i) Coughing and choking—tonsil stones that have not been removed that continue to embed themselves into the pits in the mucosa and palatine tonsils will give way to bacteria triggering tonsillitis and cause the individual to cough and, or more seriously, choke.
(ii) They are often associated with post-nasal drip.
(iii) Recurrent halitosis. The mouth is the perfect breeding ground for bacteria to multiply, and because tonsil stones are made primarily of bacteria, dead cells and mucous they can produce plaguing halitosis.
(iv) Throat closing and tightening. Tonsil stones that have been collecting for a period of time without removal and have calcified and have become sizeable can obstruct the airway thus causing breathing issues.
(v) Ear Pain. The ear and throat share nerve pathways, therefore an individual suffering with tonsil stones may experience referred ear pain.
(vi) Sore Throat and Throat Pain. Tonsil stones can become chronic and can cause soreness, irritation, or the sensation that something is caught in the throat.
(vii) Tonsil enlargement or swelling, pain when swallowing.
(viii) Pooling like an object is stuck in the throat.

Tonsil stones at times may appear as solid white material visible on the tonsil itself. Alternatively the stones may be hidden in the folds of the tonsils or in a crypt and are not visible but can still be detected by the individual. Removal of the stone will restore patient comfort and oral health.

There remains a need for an inexpensive, safe, ergonomic tool for the removal of tonsil stones and other oral debris from the oral cavity regardless of the location of the debris or stone. Various tools, electric or manual, have been used by individuals and health care providers to remove oral debris and tonsil stones. Yet there remains a need for a cost effective, sanitary, safe, device for extracting oral debris and tonsil stones from the oral cavities, mouths and throat regions of a human or mammal which is effective and minimizes the risk of harm to the oral mucosa and tissues of the oral cavity. Furthermore, there remains a need for a device which can be easily transported and carried discretely. A portable device which is lightweight and readily available for use for the removal of oral debris and stones in the oral cavity is needed for individual home use as well as use in other institutional settings.

### SUMMARY

In accordance with an aspect of the present invention, there is provided a device for removing oral debris, the device comprising:
bulb suction means;
a shaft comprising a first shaft end and a second shaft end,
the shaft cooperatively attached to the bulb suction means at the first shaft end; and
an elastomeric tip cooperatively attached to the shaft at the second shaft end.

In accordance with another aspect of the present invention, there is provided a device for removing oral debris, wherein the bulb suction means further comprises grip means.

In accordance with another aspect of the present invention, there is provided a device for removing oral debris, wherein the grip means are present on the surface of said bulb suction means.

In accordance with another aspect of the present invention, there is provided a device for removing oral debris, wherein the grip means form an integral part of the bulb suction means.

In accordance with another aspect of the present invention, there is provided a device for removing oral debris, wherein the grip means comprise a plurality of completely circumferential or incompletely circumferential ridge lines along the surface of the bulb suction means. In accordance with another aspect, the plurality of ridge lines comprises a plurality of substantially parallel lines spaced apart from one another.

In accordance with another aspect of the present invention, there is provided a device for removing oral debris, wherein the ridge lines rise from the surface of the bulb suction means to a height in the range from about 1 Rim to about 5 mm.

In accordance with another aspect of the present invention, there is provided a device for removing oral debris, wherein the bulb suction means further comprises a first bulb end and a second bulb end wherein said first bulb end comprises an opening adapted to receive said shaft.

In accordance with another aspect of the present invention, there is provided a device for removing oral debris, wherein the bulb section means further comprises a first bulb end and a second bulb end where said second bulb end comprises a recessed portion. In accordance with another aspect, the recessed portion is adapted to receive a user's thumb. In accordance with another aspect, the recessed portion comprises an indentation at the second bulb end of the bulb suction means.

In accordance with another aspect of the present invention, there is provided a device for removing oral debris, wherein the bulb suction means is substantially shaped in a shape selected from the group of shapes consisting of obconical shapes, obpyriform shapes, pyriform shapes, turbinate shapes, obelavinate shapes, pear-shaped shapes, and mushroom-shaped shapes. In accordance with another aspect, the bulb suction means is substantially obpyriform shaped.

In accordance with another aspect of the present invention, there is provided a device for removing oral debris, wherein the bulb suction means is a natural or a synthetic rubber. In accordance with another aspect, the rubber is deformable upon applying pressure to the bulb suction means.

In accordance with another aspect of the present invention, there is provided a device for removing oral debris, wherein the shaft is made from plastic. In accordance with another aspect, the shaft comprises a rigid plastic.

In accordance with another aspect of the present invention, there is provided a device for removing oral debris, wherein the shaft is translucent or transparent.

In accordance with another aspect of the present invention, there is provided a device for removing oral debris, wherein the elastomeric tip is translucent or transparent. In accordance with another aspect, the elastomeric tip is transparent.

In accordance with another aspect of the present invention, there is provided a device for removing oral debris, wherein the tip is made from silicone. In accordance with another aspect, the tip and shaft are made from silicone. In accordance with another aspect, the tip, shaft, and bulb suction means are made from silicone. In accordance with another aspect of the present invention, the entire device is made from silicone.

In accordance with another aspect of the present invention, there is provided a device for removing oral debris, wherein the bulb suction means, the shaft and the elastomeric tip are fully detachable and reattachable. In accordance with another aspect, the detachment and reattachment point between the shaft and the bulb suction means further comprises a seal means. In accordance with another aspect, said seal means prevents leakage to ensure suction is maintained in the device when in use.

In accordance with another aspect of the present invention, there is provided a device for removing oral debris, wherein the shaft is connected to the first bulb end of the bulb suction means by a snap-fit.

In accordance with another aspect of the present invention, there is provided a device for removing oral debris, the device comprising:
bulb suction means;
a shaft comprising a first shaft end and a second shaft end, the shaft cooperatively attached to the bulb suction means at the first shaft end;
an elastomeric tip cooperatively attached to the shaft at the second shaft end; and
ejection means operationally engaging the shaft at the first shaft end.

In accordance with another aspect of the present invention, there is provided a device for removing oral debris, wherein the ejection means further comprises a first ejection end and a second ejection end, wherein the first ejection end is operationally attached to the first shaft end of the shaft, and wherein the second ejection end is cooperatively attached to the first bulb end of the bulb suction means. In accordance with another aspect, the ejection means is connected to the first bulb end of said bulb suction means by a snap-fit.

In accordance with another aspect of the present invention, there is provided a device for removing oral debris, wherein the ejection means further comprises an ejection button.

In accordance with another aspect of the present invention, there is provided a device for removing oral debris, wherein the ejection means further comprises an ejection ring.

In accordance with another aspect of the present invention the opening at the tip of the shaft into which oral debris is suctioned can be of various sizes.

In accordance with another aspect of the present invention, there is provided a device for removing oral debris, the device comprising:
a bulb;
a curved shaft comprising a first shaft end and a second shaft end,
the shaft cooperatively attached to the bulb at the first shaft end; and
an elastomeric tip, said tip attached to the shaft at the second shaft end;
wherein the tip has an opening that is continuous with a second opening disposed at the first shaft end.

In accordance with another aspect of the present invention, there is provided the device, wherein the bulb further comprises grip means on an outer surface of said bulb.

In accordance with another aspect of the present invention, there is provided the device, wherein said grip means comprise at least one raised ridge present on the outer surface of said bulb.

In accordance with another aspect of the present invention, there is provided the device, wherein the bulb further comprises a first bulb end and a second bulb end, wherein said second bulb end comprises a recessed portion.

In accordance with another aspect of the present invention, there is provided the device, wherein the recessed portion is adapted to receive a user's thumb.

In accordance with another aspect of the present invention, there is provided the device, wherein the recessed portion comprises an indentation.

In accordance with another aspect of the present invention, there is provided the device, wherein said recessed portion comprises a material of greater elasticity than the rest of said bulb.

In accordance with another aspect of the present invention, there is provided a kit comprising at least two oral debris devices.

In accordance with another aspect of the present invention, there is provided a method of aspirating oral debris from an oral cavity using the device of claims 1-85, comprising the steps of:
a) placing a user's thumb in the recessed portion;
b) placing user's index and middle fingers around the bulb;
c) depressing the bulb using the thumb, while the device is still outside of the oral cavity;
d) placing the tip of the device into the oral cavity and in close proximity to the oral debris; and
e) slowly releasing the user's thumb from the bulb for creating suction.

In accordance with another aspect of the present invention, there is provided the method, wherein the user's index and middle fingers are placed around the bulb in contact with the grip means.

In accordance with another aspect of the present invention, there is provided a method of expelling oral debris from an oral cavity using the device of claims 1-85, comprising the steps of:

a) placing a user's thumb in the recessed portion;

b) placing user's index and middle fingers around the bulb;

c) placing the tip of the device into the oral cavity; and

d) depressing the bulb using the thumb and expelling air to dislodge the oral debris.

In accordance with another aspect of the present invention, there is provided a method of plucking oral debris from an oral cavity using the device of claims 1-85, comprising the steps of:

a) pressing the tip around an area of the oral cavity where the oral debris is located;

b) dislodging the oral debris by pressing or pushing the oral debris; and

c) removing the oral debris after it has been dislodged.

In accordance with another aspect of the present invention, there is provided a method of exploring an oral cavity using the device of claims 1-85, comprising in sequence the steps of:

a) touching the tip against one side of the oral cavity;

b) touching the tip against a second side of the oral cavity; and

c) advancing the tip deeper within the oral cavity.

In accordance with another aspect of the present invention, there is provided a bulb comprising:
a first and a second end;
means for attaching the bulb to a shaft disposed at the first end;
a recessed portion adapted to receive a thumb disposed at the second end; and
grip means disposed on an outer surface of the bulb.

In accordance with another aspect of the present invention, there is provided the bulb, wherein the grip means is raised on the outer surface of the bulb to permit a user to sense the presence of the grip means when fingers of the user are in contact with the grip means.

In accordance with another aspect of the present invention, there is provided the bulb, wherein the recessed portion comprises an elastomeric material, different from the material of the bulb.

In accordance with another aspect of the present invention, there is provided the bulb, wherein the elastomeric material has a greater elasticity than the material of the bulb.

### BRIEF DESCRIPTION OF THE FIGURES

Other and further advantages and features of the invention will be apparent to those skilled in the art from the following detailed description taken together with the accompanying Figures.

For the purpose of illustrating the invention, there is shown in the Figures embodiments which are presently preferred; it being understood, however, that other embodiments are possible and would be understood or appreciated by a person skilled in the art. The invention is not limited only to the precise arrangements and instrumentalities shown as these Figures.

The Figures only exemplify particular embodiments of the invention and other embodiments would be understood to be operable within the scope of the invention set forth in the specification when read and understood as a whole.

In the accompanying Figures like reference numerals refer to like parts throughout, in which:

FIG. 1 illustrates a top perspective view of an embodiment of the present invention.

FIG. 2 illustrates a bottom perspective view of an embodiment of the present invention.

FIG. 3 illustrates a right view of an embodiment of the present invention.

FIG. 4 illustrates a left side view of an embodiment of the present invention.

FIG. 5 illustrates a top view of an embodiment of the present invention.

FIG. 6 illustrates a front view of an embodiment of the present invention.

FIG. 7 illustrates a bottom view of an embodiment of the present invention.

FIG. 8 illustrates a back view of an embodiment of the present invention.

FIG. 9 illustrates a top view of an embodiment of the present invention, identifying a line of cross-section A-A.

FIG. 10 illustrates a cross-sectional view of an embodiment of the present invention along the line of cross-section A-A.

FIG. 11 illustrates a side view of an embodiment of the present invention, illustrating hand positioning when in use.

FIG. 12 illustrates a side view of an embodiment of the present invention illustrating deformation of a portion of the bulb and also the hand positioning when in use.

FIG. 13 illustrates a side view of an embodiment of the present invention as it enters an oral cavity.

FIG. 14A illustrates a perspective view of an embodiment of the present invention comprising a 2 mm diameter hole size at the tip opening.

FIG. 14B illustrates a perspective view of an embodiment of the present invention comprising a 3 mm diameter hole size at the tip opening.

FIG. 14C illustrates a perspective view of an embodiment of the present invention comprising a 4 mm diameter hole size at the tip opening.

FIG. 15 illustrates a top perspective view of a second embodiment of the present invention.

FIG. 16 illustrates a rear perspective view of a second embodiment of the present invention.

FIG. 17 illustrates a right view of a second embodiment of the present invention.

FIG. 18 illustrates a left side view of a second embodiment of the present invention.

FIG. 19 illustrates a top view of a second embodiment of the present invention.

FIG. 20 illustrates a front view of a second embodiment of the present invention.

FIG. 21 illustrates a bottom view of a second embodiment of the present invention.

FIG. 22 illustrates a back view of a second embodiment of the present invention.

FIG. 23 illustrates a top view of a second embodiment of the present invention, identifying a line of cross-section B-B.

FIG. 24 illustrates a cross-sectional view of a second embodiment of the present invention along the line of cross-section B-B.

FIG. 25 illustrates a side view of a second embodiment of the present invention, illustrating hand positioning when in use.

FIG. 26 illustrates a side view of a second embodiment of the present invention illustrating the deformation of a bulb portion and also the hand positioning when in use.

FIG. 27 illustrates a side view of a second embodiment of the present invention as it enters an oral cavity.

FIG. 28 illustrates a perspective view of a third embodiment of the present invention.

FIG. 29 illustrates a top perspective view of a fourth embodiment of the present invention.

FIG. 30 illustrates a bottom perspective view of a fourth embodiment of the present invention.

FIG. 31 illustrates a right view of a fourth embodiment of the present invention.

FIG. 32 illustrates a left side view of a fourth embodiment of the present invention.

FIG. 33 illustrates a top view of a fourth embodiment of the present invention.

FIG. 34 illustrates a front view of a fourth embodiment of the present invention.

FIG. 35 illustrates a bottom view of a fourth embodiment of the present invention.

FIG. 36 illustrates a back view of a fourth embodiment of the present invention.

FIG. 37 illustrates a front perspective view of a fifth embodiment of the present invention.

FIG. 38 illustrates a back perspective view of a fifth embodiment of the present invention.

FIG. 39 illustrates a side view of a fifth embodiment of the present invention.

FIG. 40 illustrates a top view of a fifth embodiment of the present invention.

FIG. 41 illustrates a perspective view of an embodiment of a bulb of the present invention.

FIG. 42 illustrates a side view of an embodiment of an ejector body of the present invention.

FIG. 43 illustrates a cross-sectional view of an embodiment of a bulb connected to an embodiment of an ejector body of the present invention.

FIG. 44 illustrates a cross-sectional view of an embodiment of a bulb connected to another embodiment of an ejector body of the present invention.

FIG. 45 illustrates a partial expanded view of a sixth embodiment of the present invention, with tip detached.

FIG. 46 illustrates a perspective view of an embodiment of an ejector of the present invention.

FIG. 47 illustrates a top perspective view of a seventh embodiment of the present invention.

FIG. 48 illustrates a bottom perspective view of a seventh embodiment of the present invention.

FIG. 49 illustrates a right view of a seventh embodiment of the present invention.

FIG. 50 illustrates a left side view of a seventh embodiment of the present invention.

FIG. 51 illustrates a top view of a seventh embodiment of the present invention.

FIG. 52 illustrates a front view of a seventh embodiment of the present invention.

FIG. 53 illustrates a bottom view of a seventh, embodiment of the present invention.

FIG. 54 illustrates a back view of a seventh embodiment of the present invention.

FIG. 55 illustrates a top view of a seventh embodiment of the present invention, identifying a line of cross-section A-A.

FIG. 56 illustrates a cross-sectional view of a seventh embodiment of the present invention along the line of cross-section A-A.

FIG. 57 illustrates a side view of another embodiment of a bulb of the present invention.

FIG. 58 illustrates a front view of another embodiment of a bulb of the present invention.

FIG. 59 illustrates a side view of an embodiment of a shaft and tip of the present invention.

FIG. 60 illustrates a side view of an eighth embodiment of the present invention.

FIG. 61 illustrates a perspective view of a ninth embodiment of the present invention in use.

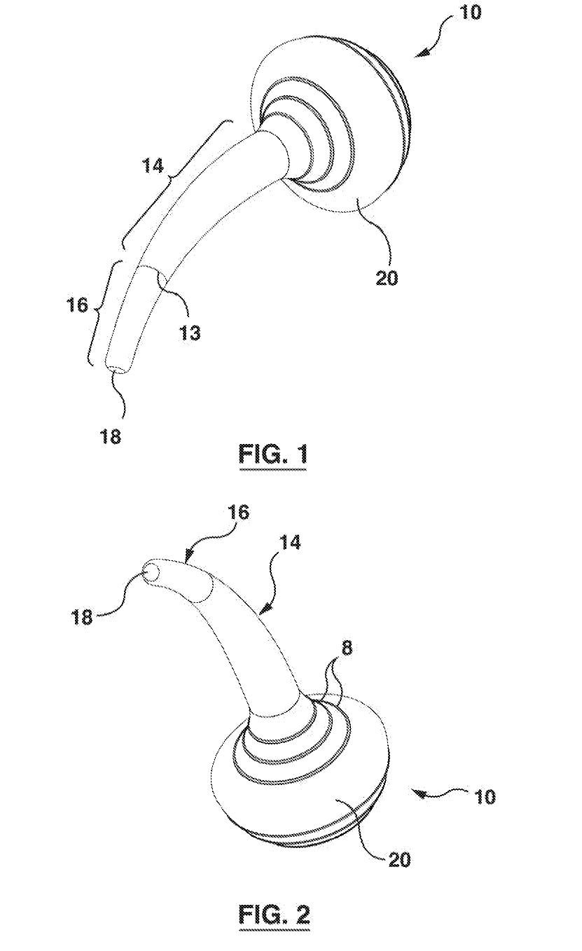

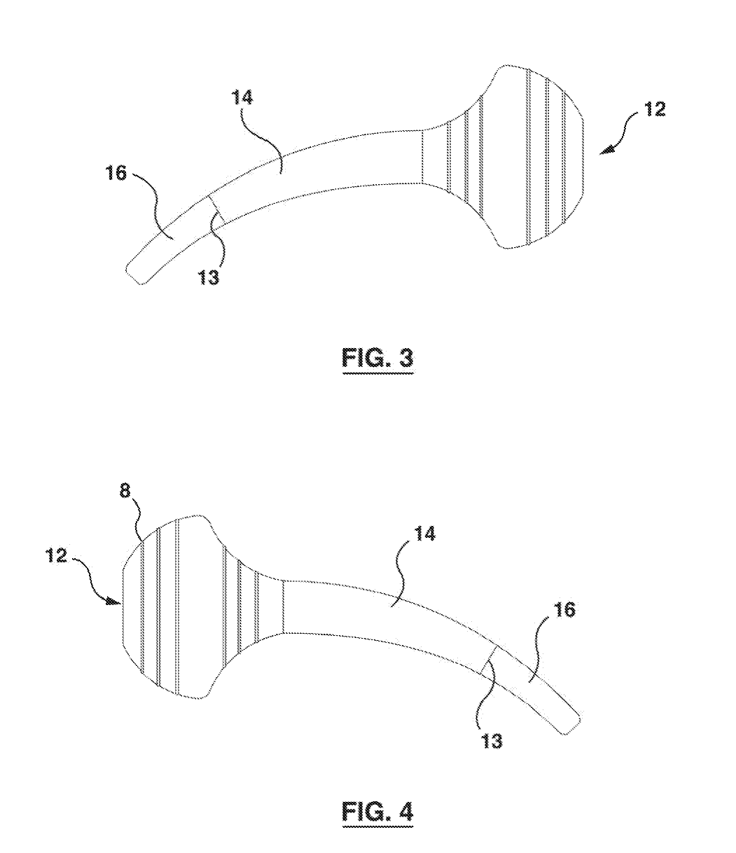

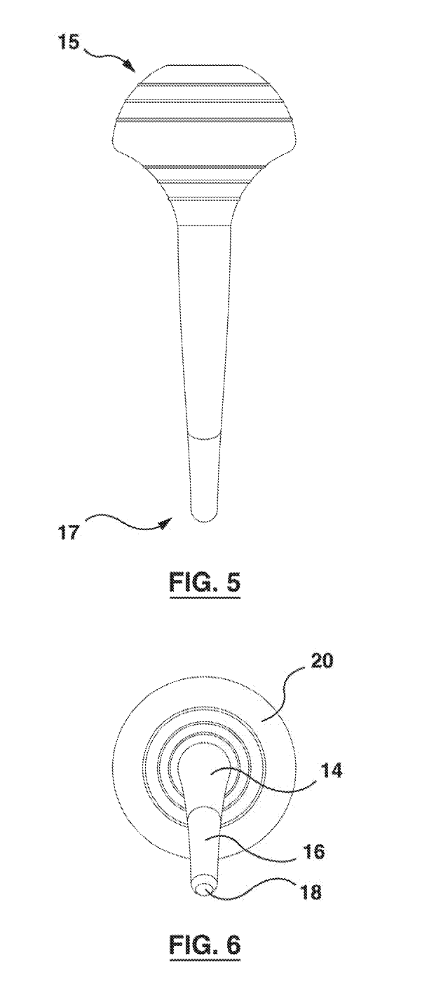

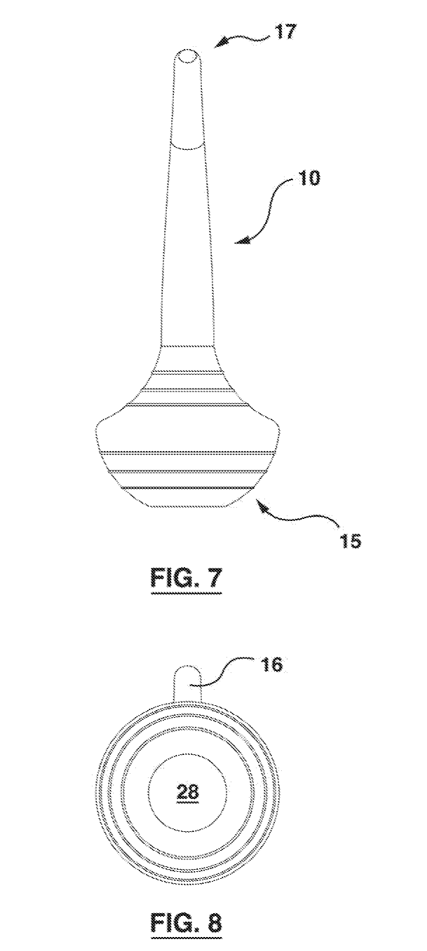

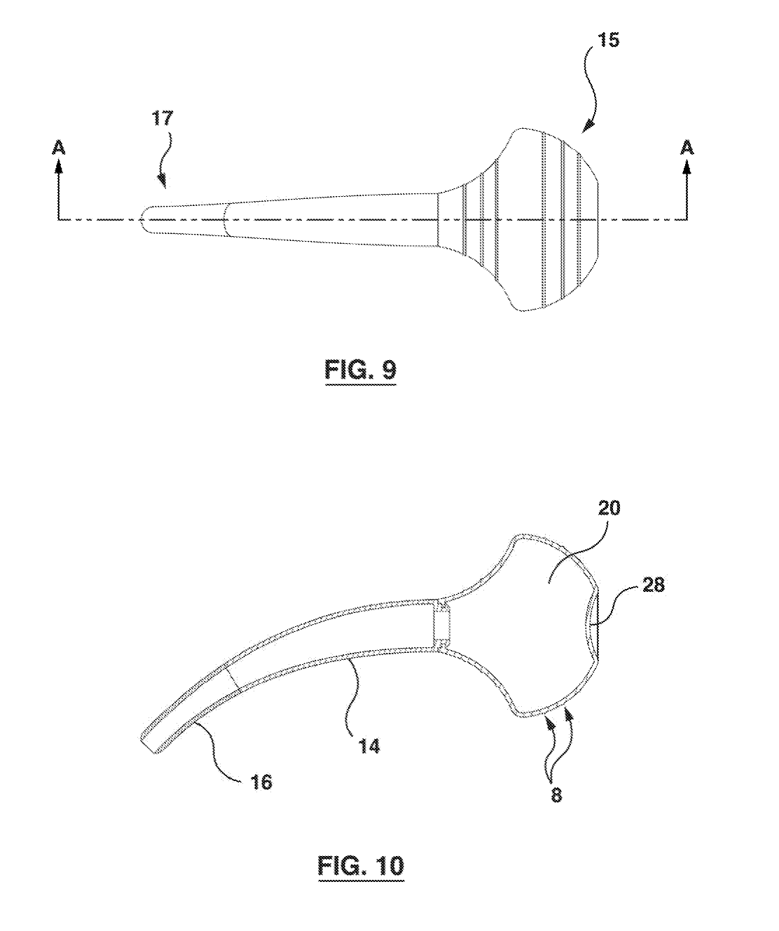

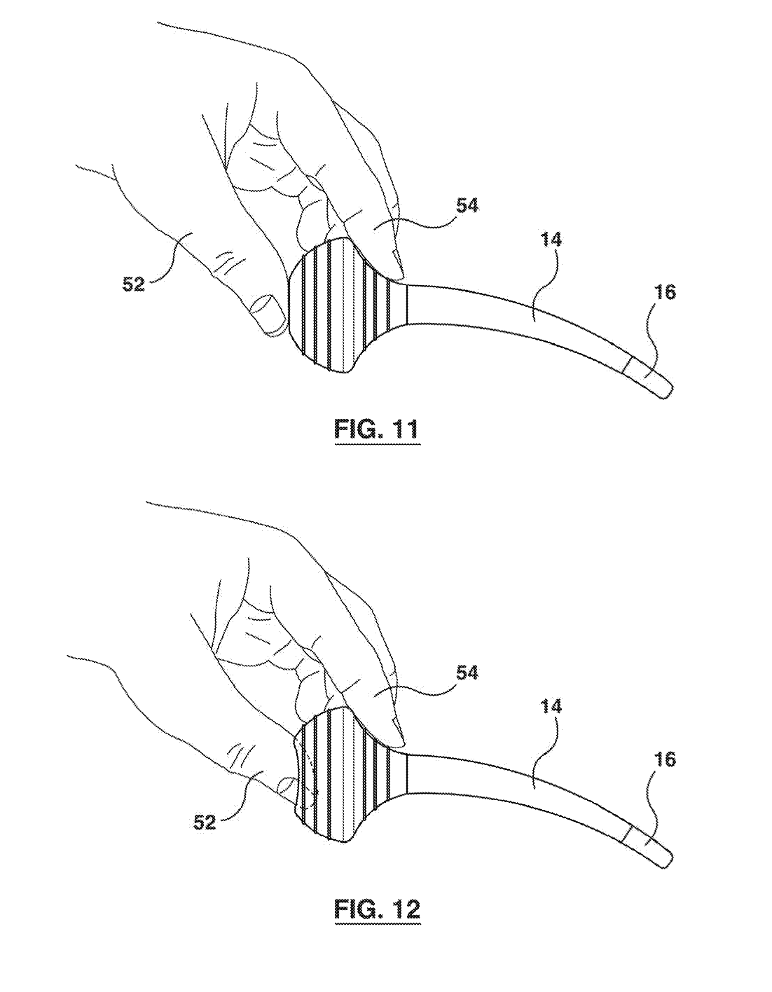

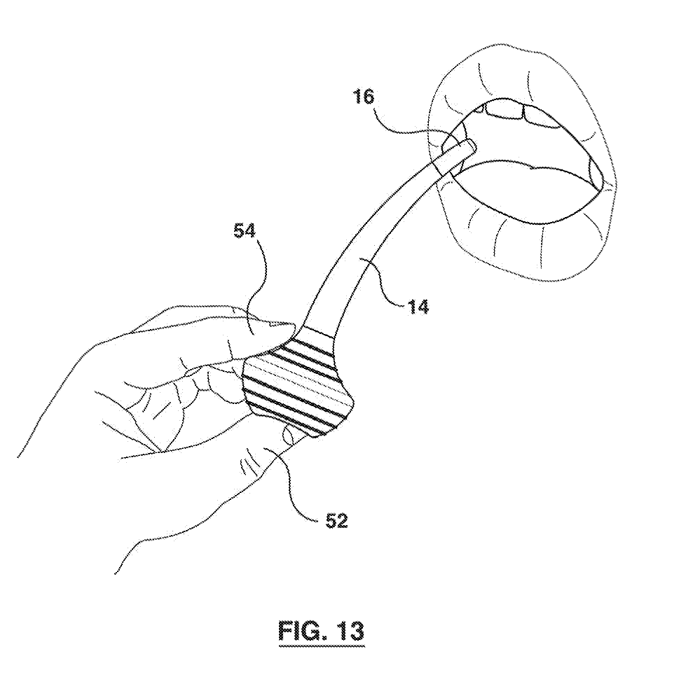

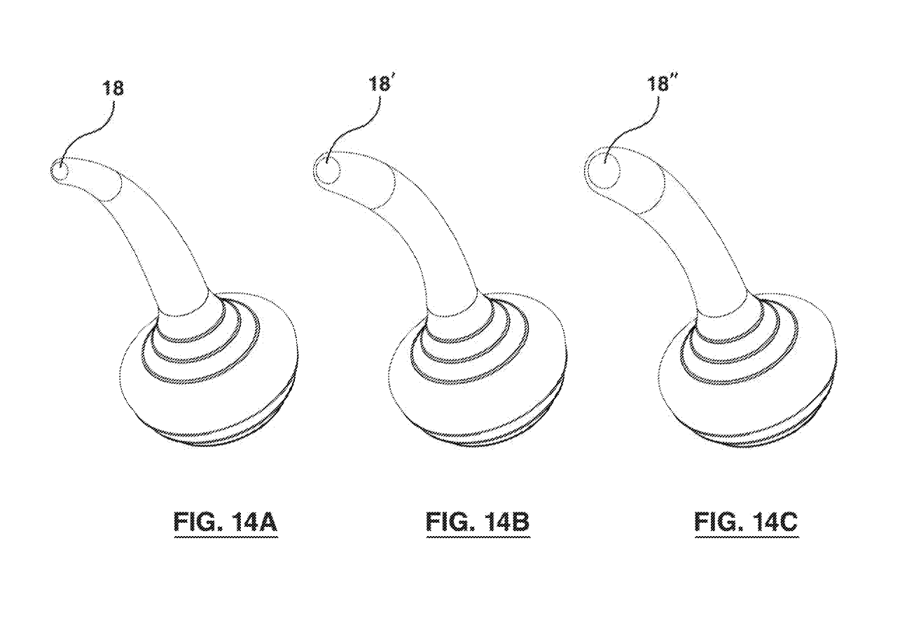

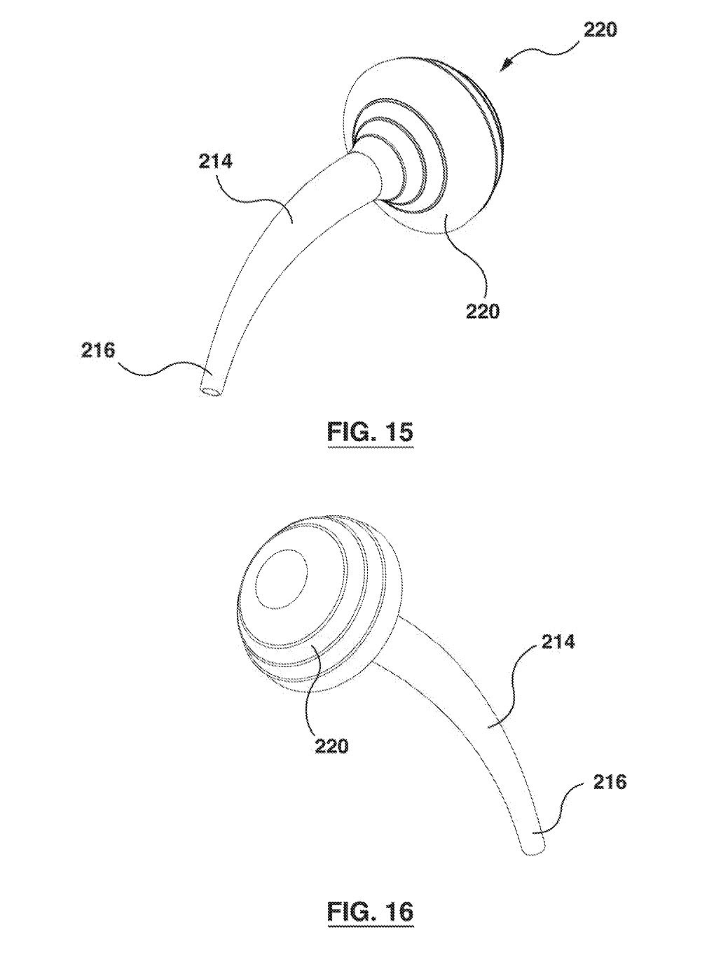

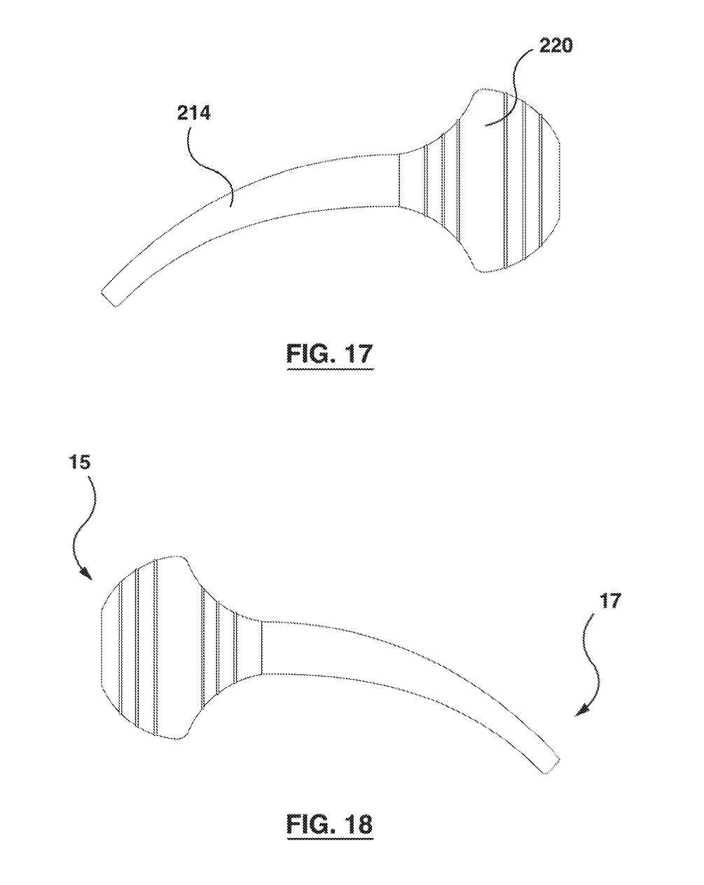

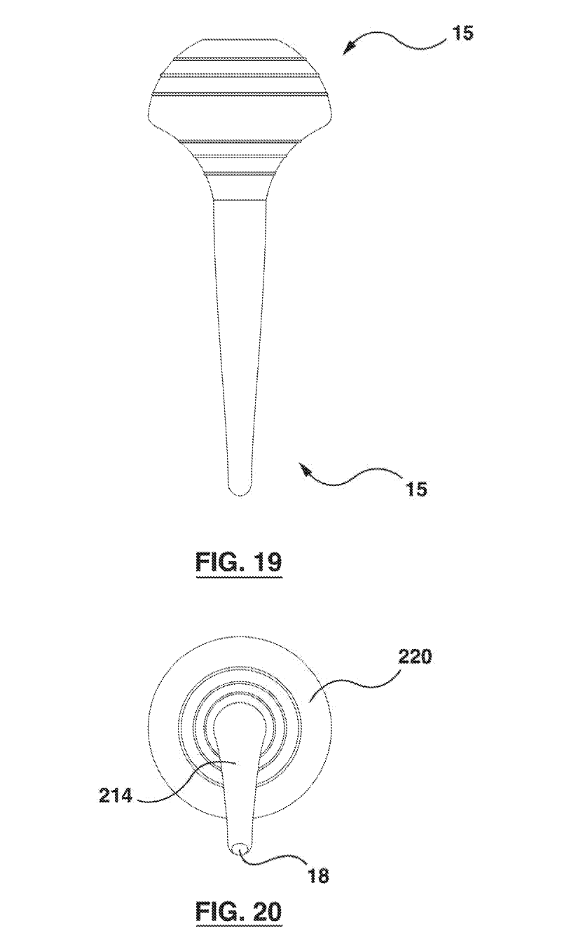

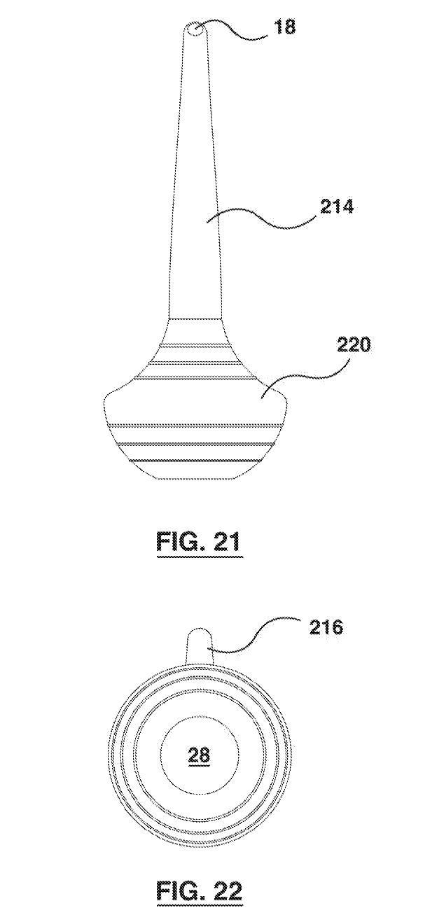

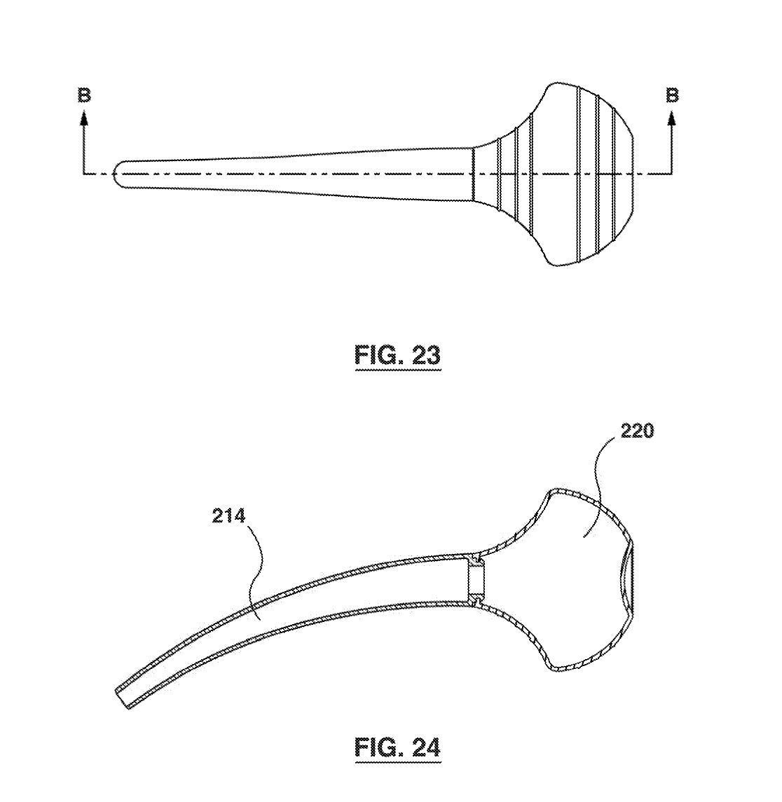

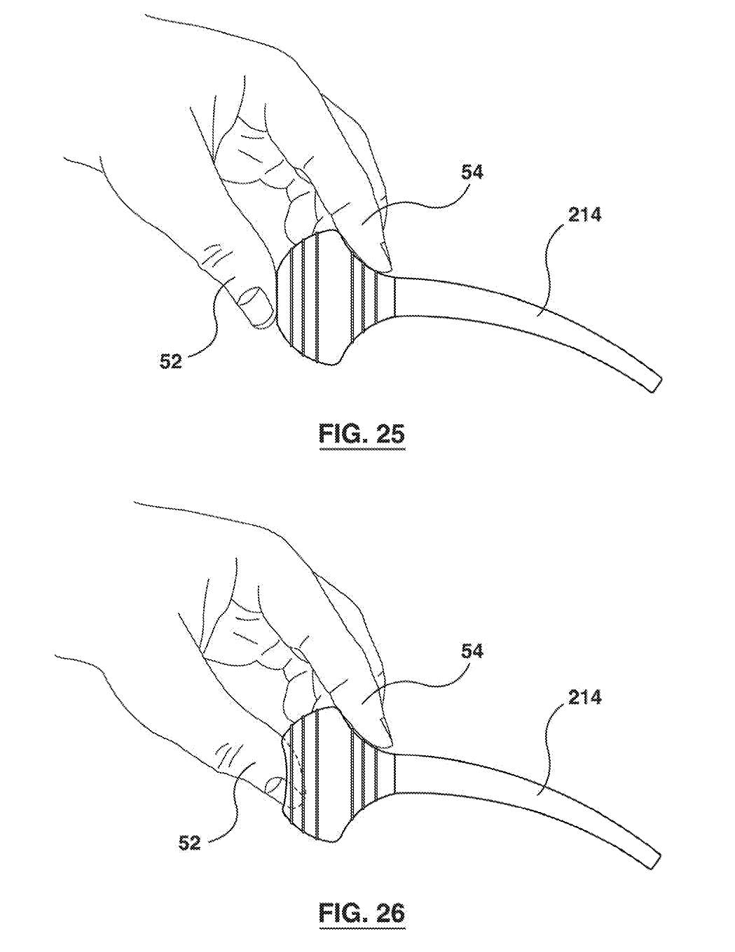

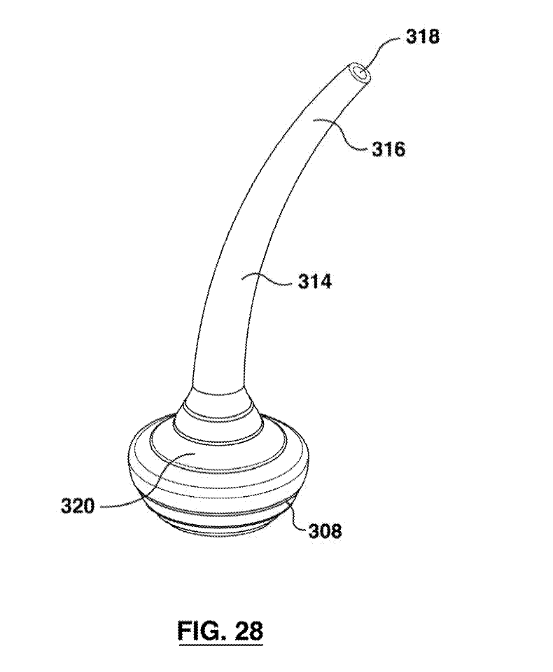

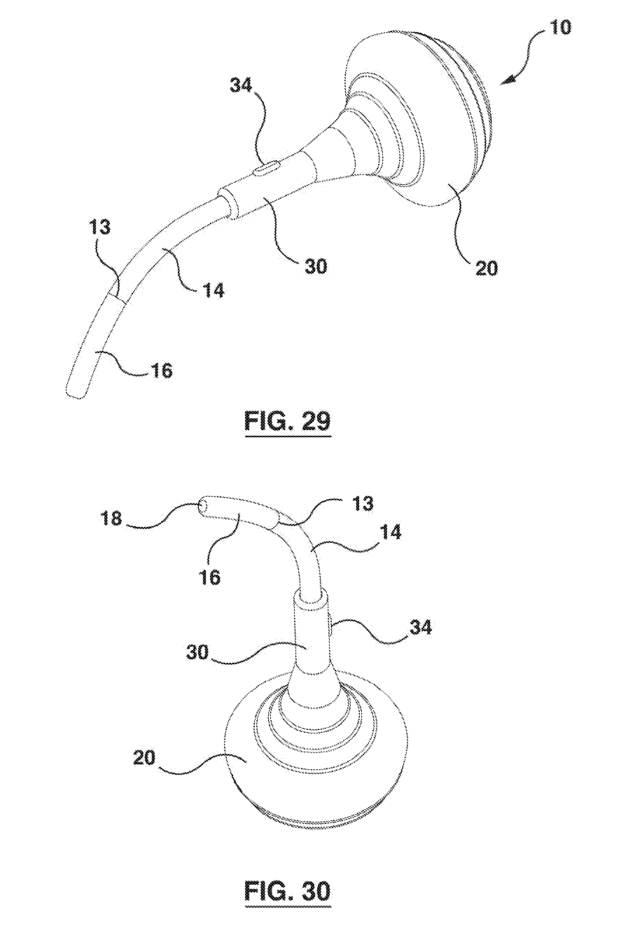

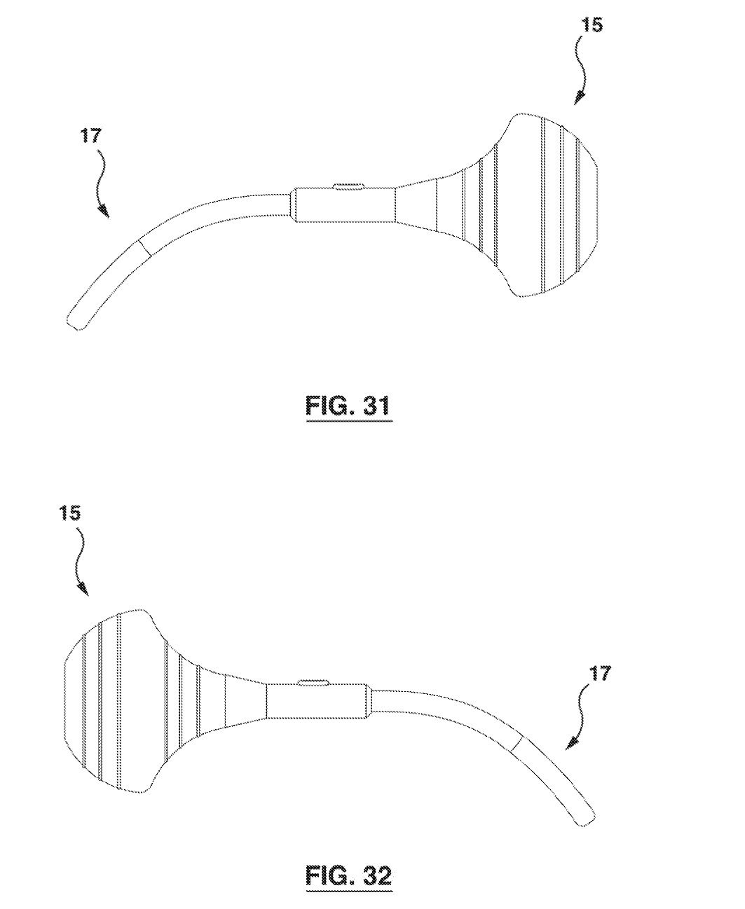

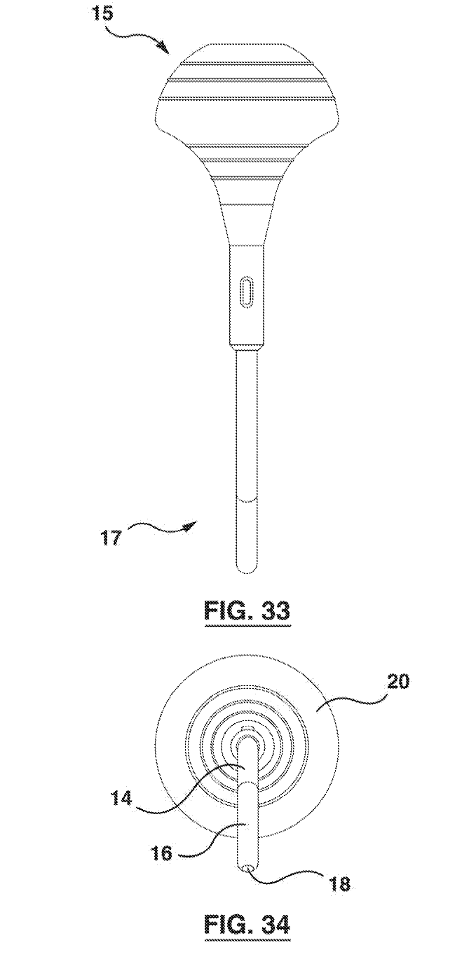

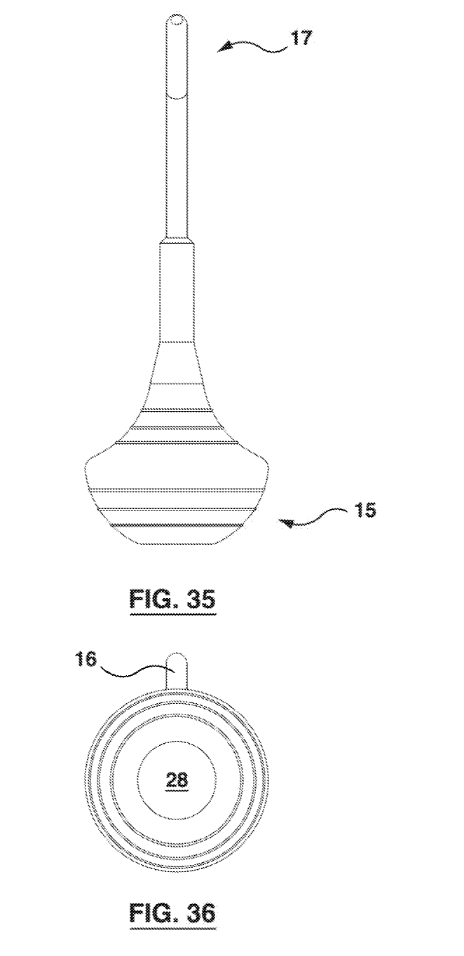

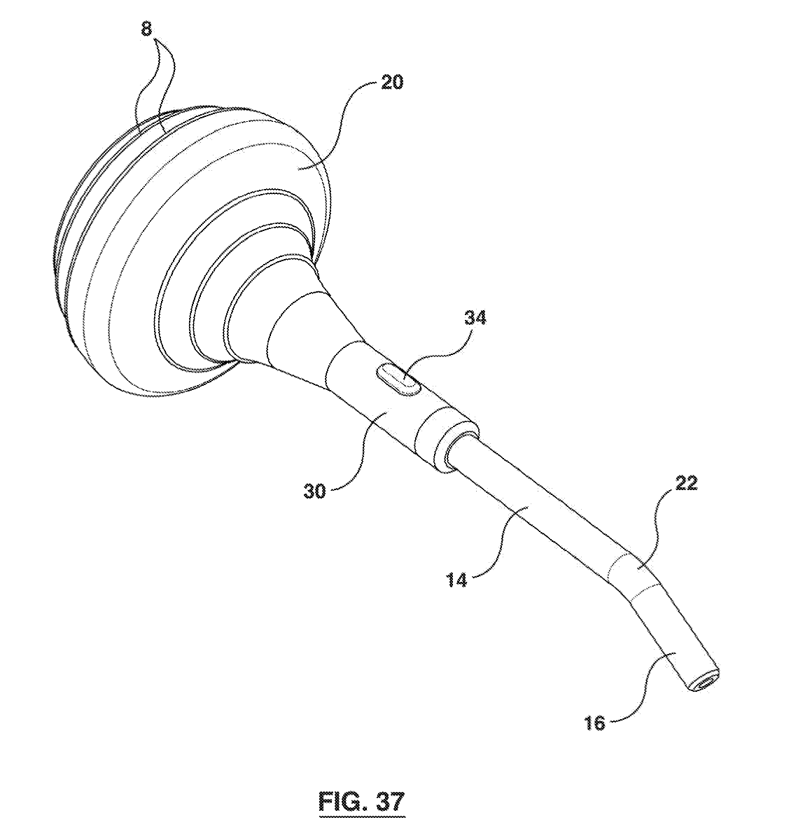

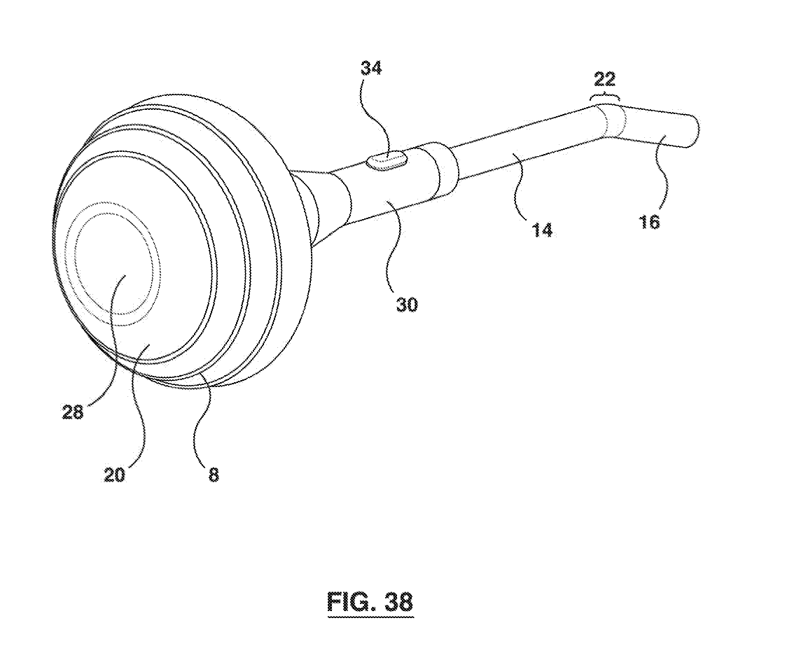

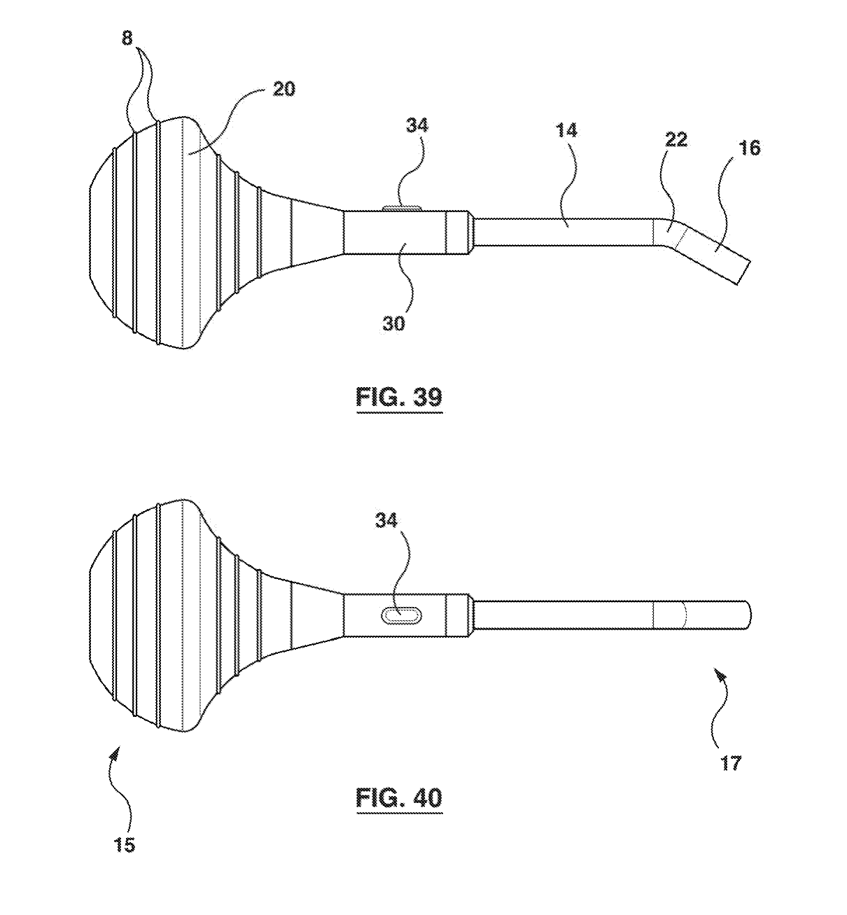

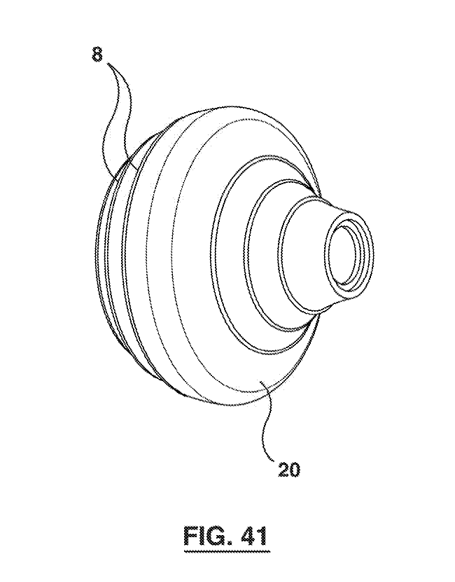

A first embodiment of the device is illustrated in FIGS. 1 to 10. The manner in which this embodiment and other similar embodiments of the device is held and used is exemplified, for example, in FIGS. 11 to 13. FIGS. 14A to 14B illustrate various diameter sizes for the hole opening in the tip portion. A second embodiment of the device is illustrated in FIGS. 15 to 24, and the manner in which this embodiment of the device is held and used is exemplified in FIGS. 25 to 26. A third embodiment of the device is illustrated in FIG. 28. A fourth embodiment of the device with an ejector body is illustrated in FIGS. 29 to 36. A fifth embodiment of the device with an ejector body and an angled curvature between a shaft portion and a tip portion is illustrated in FIGS. 37 to 40.

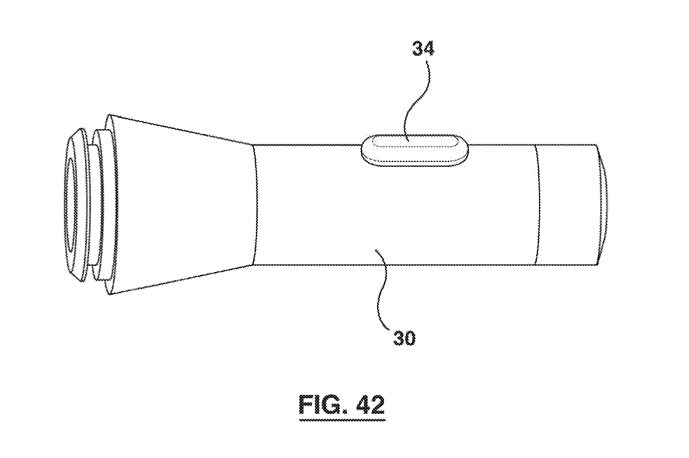

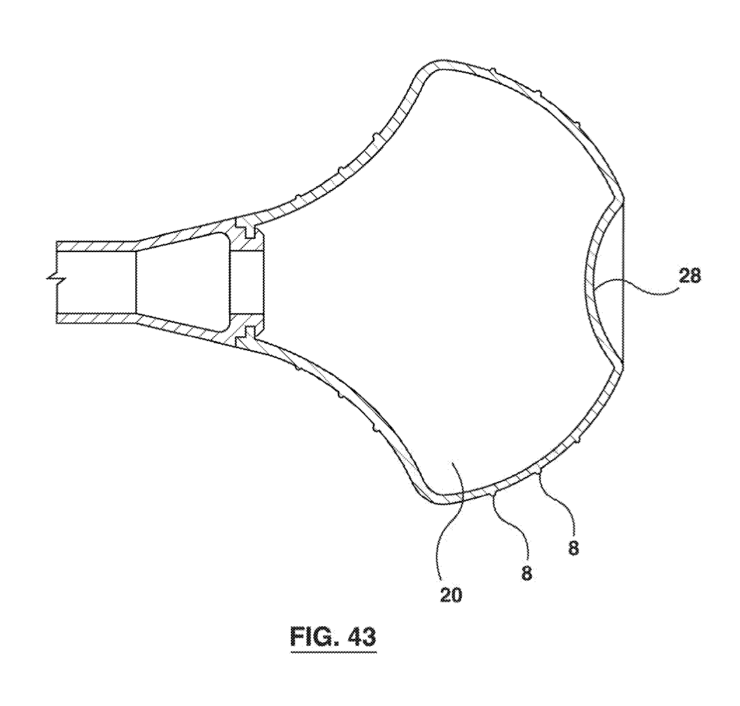

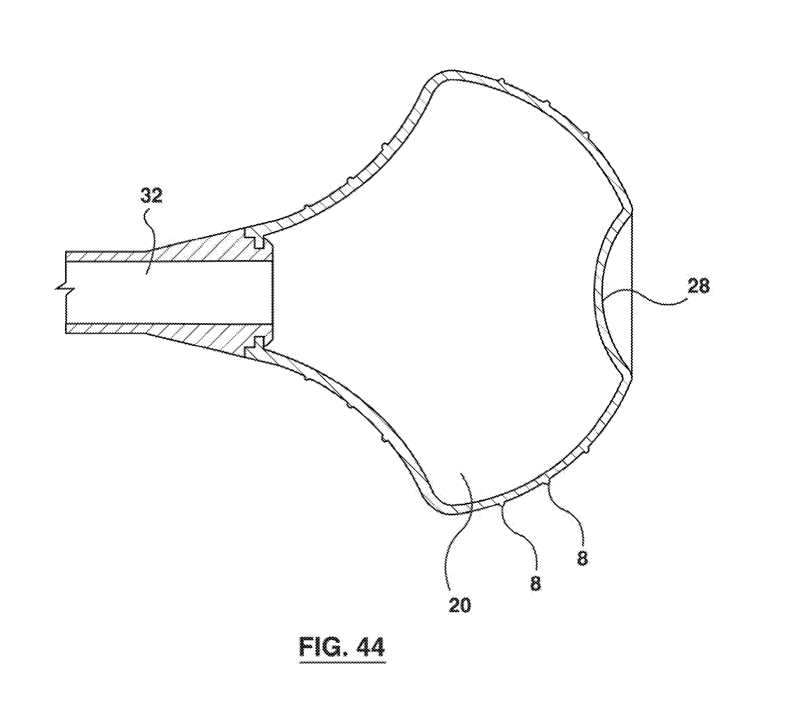

An embodiment of a bulb portion is illustrated in FIG. 41 and an embodiment of an ejector body is illustrated in FIG. 42. FIG. 43 illustrates, in cross-section, the bulb and ejector body connected together. FIG. 44 also illustrates, in cross-section, the bulb and another embodiment of an ejector body connected together.

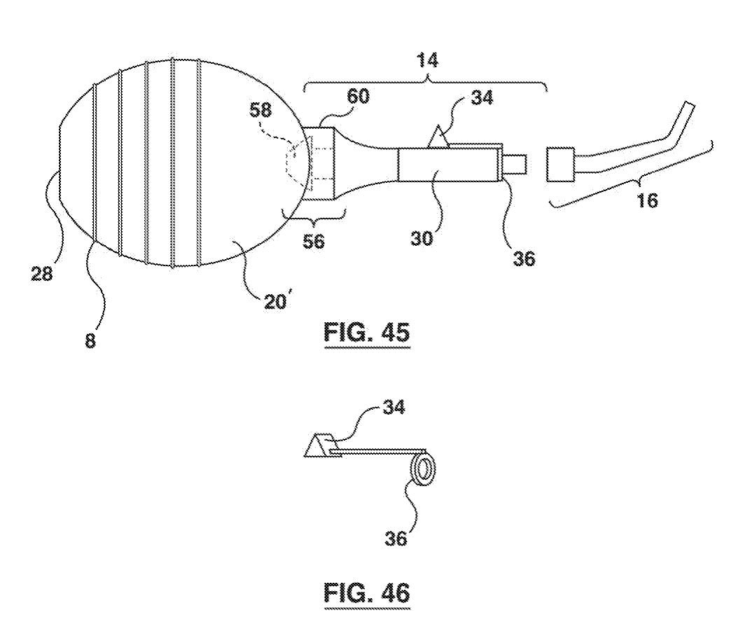

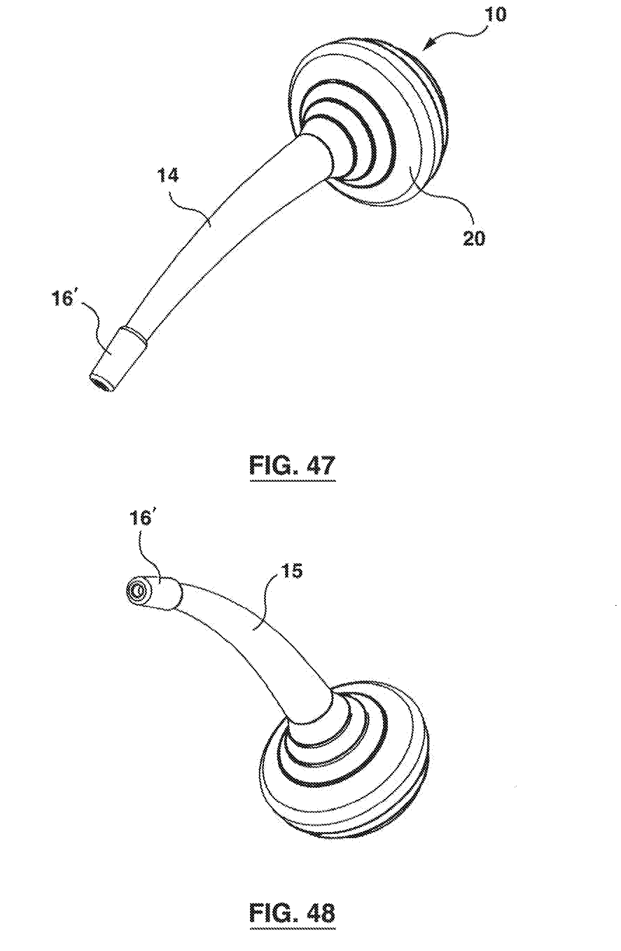

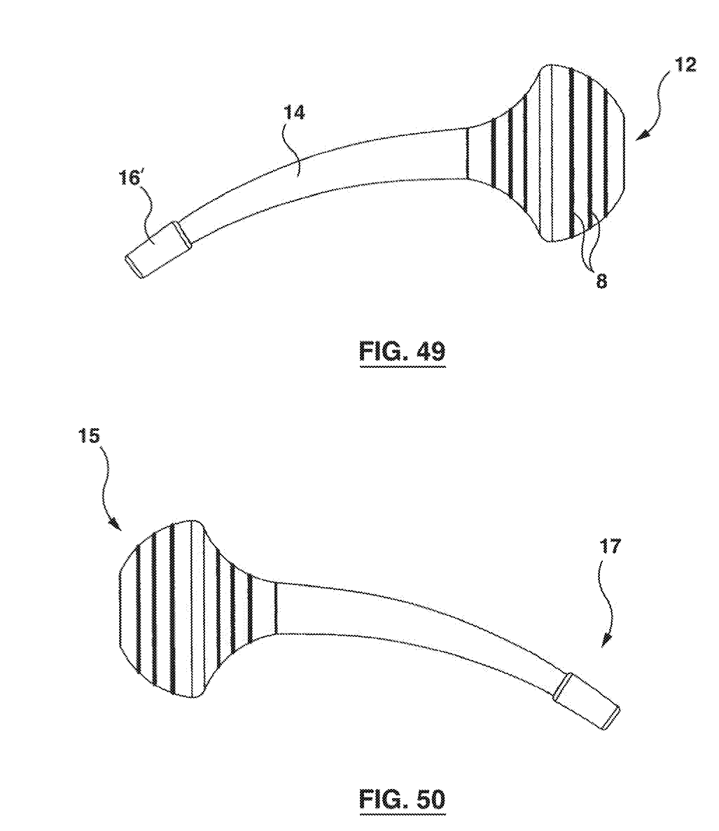

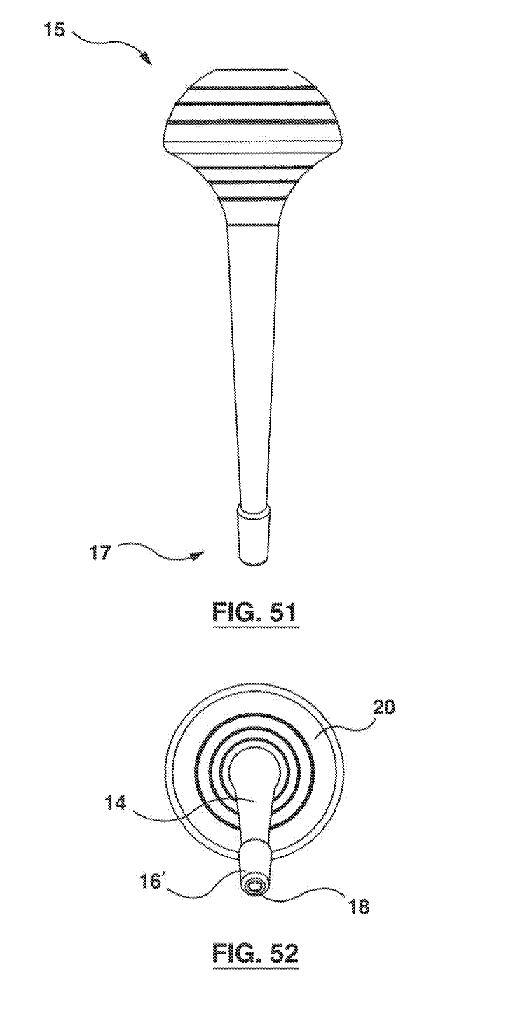

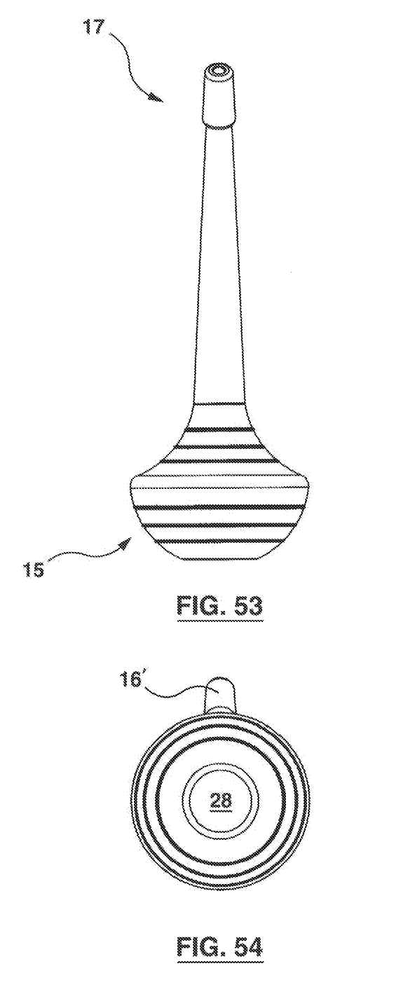

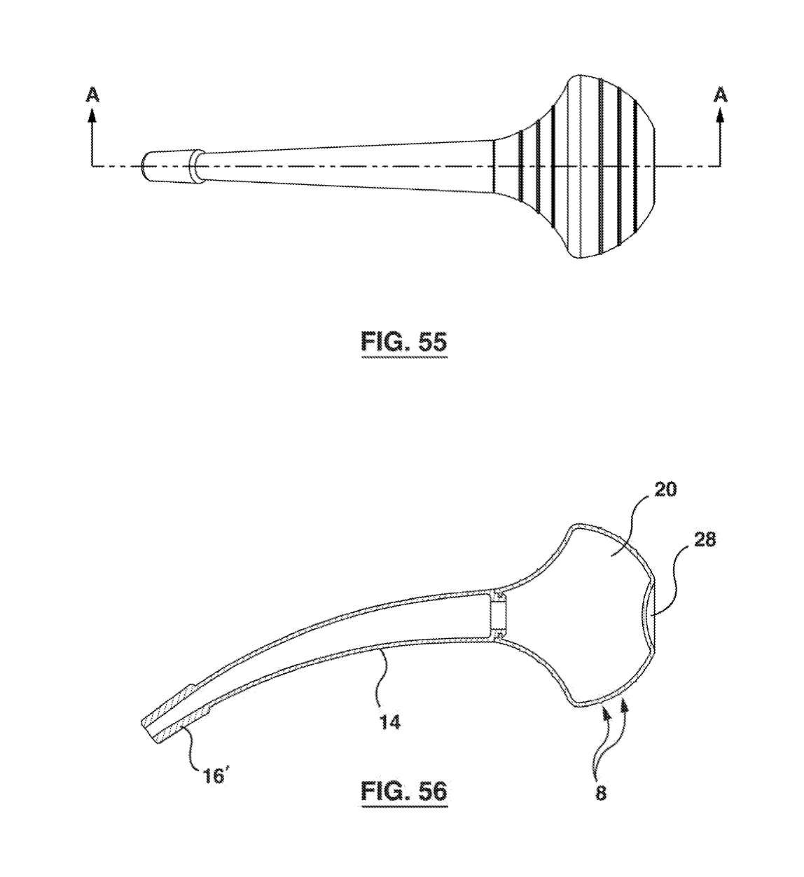

A sixth embodiment of the device is illustrated in FIG. 45. This embodiment comprises an ejector portion which is shown in FIG. 46. A seventh embodiment of the device is illustrated in FIGS. 47 to 56.

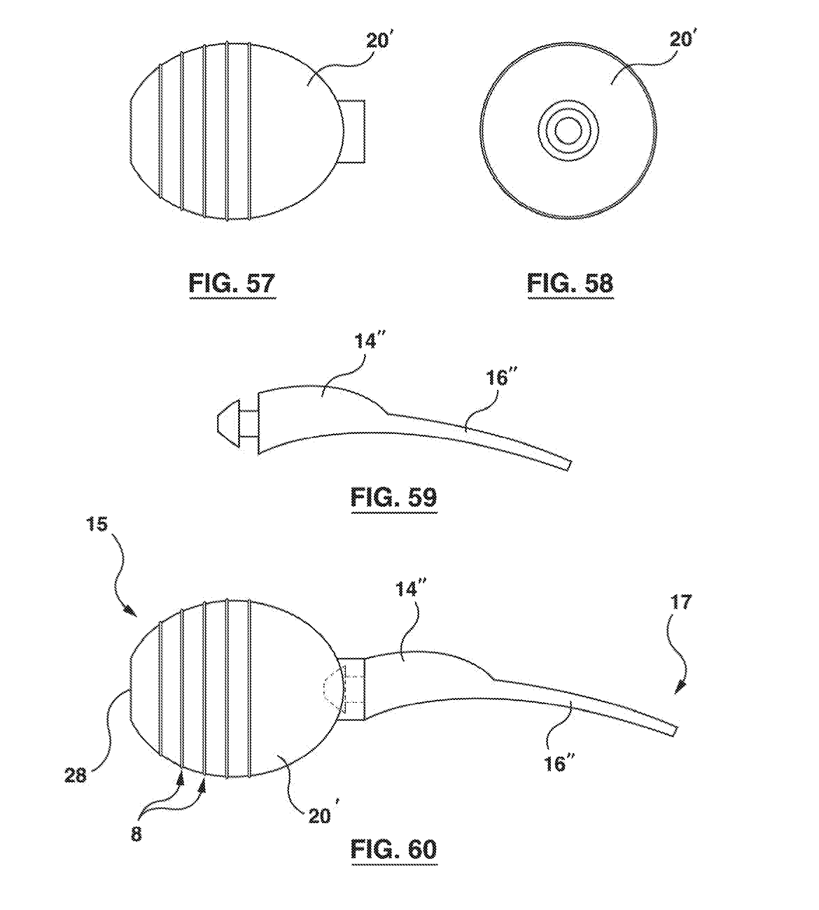

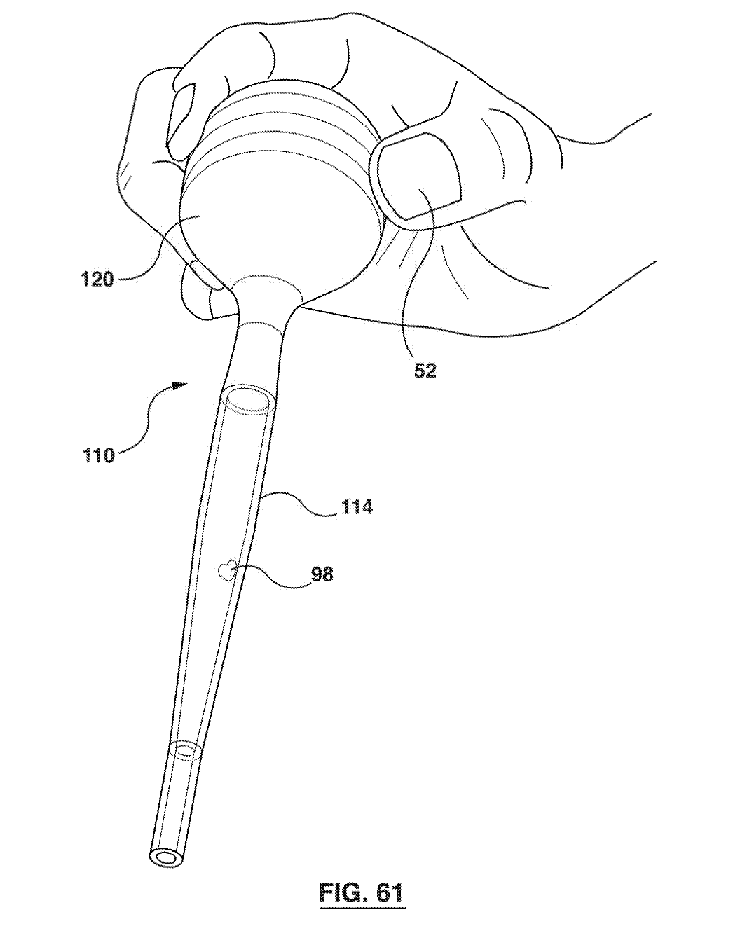

Another embodiment of a bulb portion is illustrated in FIGS. 57 and 58, and another embodiment of a shaft and tip combination is illustrated in FIG. 59. An eighth embodiment of the device is illustrated in FIG. 60. A ninth embodiment of the device is illustrated in FIG. 61.

### DETAILED DESCRIPTION

In accordance with an aspect of the invention, there is provided an oral debris extractor to explore, examine, or remove oral debris from the mouth of a mammal including removal of tonsil stones from the mouth and oral cavity of a mammal. In this specification, the word “patient” is used and intended to include humans and non-human mammals. A patient may also be a human who uses the oral debris extractor as part of their self-care and need not be under the care of a medical professional although they may be. Similarly, in the case of companion animals and pets, such as a dog, it may be the owner who is using the oral debris extractor to treat their pet and need not be a veterinary professional or assistant. Therefore the term “patient” is defined broadly in this specification and includes individuals in circumstances for self-care in the case of a person and owner care in the case of a pet or companion animal even if not under the supervision of a medical or veterinary professional.

The device may be manufactured as one piece or it may be assembled from separate pieces. An advantage of a manufacture as a single unitary piece is that there are no parts which may detach during use, for example. The mode of manufacture will vary depending on the intended method of manufacture and the techniques of manufacture and modifications required to most cost effectively manufacture the device. It is understood that the oral debris extractor of the present invention may be manufactured as a single, molded, unitary device which is one piece or it could be manufactured in parts which are then assembled as part of the manufacturing process. The selection of appropriate manufacture options would be known to a person skilled in the art and may also vary depending on the embodiment.

The least preferred embodiment is to sell the three main pieces (the bulb, the shaft, and the tip) separately for home assembly which although these may work, once assembled, there is an increased risk of a reduced suction force because of possible gaps or leaks at the junctions between the parts. If the device is manufactured in parts and assembled before sale, seals can be placed at the junctions during manufacturing to reduce the likelihood of leaks and detachments.

Advantages of the device include that it is a manually operated device with no pointed or other sharp edges which could harm the patient when in use. The curved shaft is also less likely to cause harm to a patient or individual when in use.

The curved shaft portion of the device provides benefits for the use of the device over devices with straight shafts. However, one skilled in the art would appreciate that the benefits of the shaft would be appreciated with many different types of bulbs and bulb embodiments. A curved shaft provides greater comfort for the patient. It fits into the oral cavity better and therefore allows for exploration of the oral cavity. In a preferred embodiment the shaft is made from a material which is sufficiently sturdy to permit it to aspirate, explore, assess, expel air and swipe the inside of the oral cavity; yet the material is gentle to the skin because it is elastomeric in nature that the tissues and oral cavity are not harmed by the action of the device when used within the oral cavity of a human or mammal. In accordance with national regulations for a specific country, the material selected must meet the standards for human or veterinary use.

The curve in the shaft may be likened to the bend in a finger and the device embodying a curved shaft may be used as a finger and as a substitute for a finger swipe. The curvature of the shaft has numerous advantages.

A curved shaft is advantageous because the shape facilitates exploration of the oral cavity. The patient also feels less trepidation when they see the device that will be used. It reduces the feeling of being stabbed or the risks of an unwanted sudden movement that could cause harm to the patient or individual. Self use is facilitated because the hand does not have to be so far away from the face as may be necessary with a straight shafted device. The curved device can gently touch the interior of the oral cavity to increase acceptance and facilitate reduction in the natural gag reflex that can occur when something straight is placed into the mouth. Intuitively the curved shaft is safer because of how it is more suited to fit into an oral cavity.

It is very important that the mucosa of the lining of the oral cavity is not punctured as this could lead to infection and even the need for surgical correction.

In use, the device is more likely to be accepted by the patient because of the material out of which it is made as well as the curvature of the shaft. It is known by a person skilled in the art that the gentle touching within the cavity, such as the tongue, or the inside of the cheeks facilitates acceptance of the use of the device in their mouth and they are able to relax more during use. Also the interior of the oral cavity becomes habituated to the device with this relaxation and sensory acceptance of the device inside the oral cavity. While nothing will fully prevent gagging these benefits help to maximize the functionality of the device as it is easier to locate the debris and/or stones and remove the debris and/or stones. All of these benefits are connected to the curve in the shaft as well as the material out of which the shaft is made. One skilled in the art would know that many materials are possible which would not damage the mouth yet be firm enough to act as an oral debris extractor. Suitable materials include rubber and silicone.

While in a preferred embodiment the device may be molded all at once, it is appreciated that individual parts may be separately molded and then connected one to the other. Embodiments of both of these approaches are illustrated and described in this specification. In a preferred embodiment the entire device is disposable as a recyclable object. A preferred embodiment of the present invention is fully disposable after each use, however other embodiments are possible and may be practicable in a suitable environment such as a medical facility where sterilization tools such as UV light and autoclaves are available. It is appreciated that in other embodiments, such as an embodiment comprising a removable tip or ejected tip, the tip portion alone may be disposable after each use and a new tip used the next time. In some instances the entire device in its entirety may be autoclavable and in other instances the device without the tip (which is a separate sanitary item) is autoclavable. It is recognized and understood that there are a variety of autoclaves that one skilled in the art would know are appropriate for the material used. For example http://www.glstpes.com/pdf/papers/Thermoplastic%20Elastomers%20in%20Medical%20Devices.pdf describes and discusses the use of thermoplastic elastomers in medical devices. In another example, thermoplastic elastomers including innovative TPE solutions may be used. In a preferred embodiment, the shaft and tip are made from a polypropylene such as (SR549M PolyPro) because of the super clarity properties associated with this material and it also is known to meet FDA, TSCA and DSL chemical requirements. In a preferred embodiment, the bulb portion is made from a thermoplastic elastomer—TPE being a recyclable material.

The device of the present invention is portable, cost effective, ergonomic device which can be sold in a sanitary condition for single use. It could be sold alone or packaged in groups of more than one device for sale. Different embodiments could also be packaged together as sets or kits, for example different sizes of openings at the tip could be grouped in a set for sale and then the user would select the version most appropriate for the patient and/or the nature of the debris which has accumulated.

When deciding whether to use a tip with a larger or smaller opening for aspiration it is appreciated that the size of the debris is one consideration. It is also appreciated that a larger opening may remove the debris more quickly which, in some more sensitive patients, may be more painful. Accordingly the selection of tip opening size has both a practical reason and a personal choice reason which could come into play when making this decision. Even for larger stones, a smaller tip opening may still be preferred by the patient. Tip openings may range but typically will fall into a range of from about 0.75 mm to about 4.5 mm. With a more preferred range from 2 mm to 4 mm and a most preferred diameter of the opening in the tip at 2 mm. A kit with a set of instructions and a selection of devices could be sold in a set to accommodate the needs of different persons or circumstances.

The combination of a recessed depressible portion in the bulb, in a preferred embodiment, facilitates stability and control of the device during use as does the overall selection of a rubber or silicone material of manufacture. The material allows greater control of the bulb by the user when suction is applied. This applies whether the unit has a separately identified portion on the bulb or not A recessed portion for thumb placement enhances secure grip as well as control over use of the bulb when applying and releasing the suction.

Initially, the device may be used for exploration of the oral cavity of a patient and particular tissues or glands to determine the presence or absence of oral debris and the nature of the oral debris in order to first assess the patient before any treatment decision is made. Although the stones may be hidden in the folds or in the crypts of the tonsils or glandular tissues in the oral cavity they can still be safely detected by exploring the oral cavity with the oral extractor and then removed.

In accordance with another aspect of the device, a-recessed depressible and/or deformable portion of the bulb, is provided on a surface of the bulb preferably at the distal end of the bulb. The distal end of the bulb is opposite the proximal end of the bulb. The proximal end of the bulb meets the shaft. The recessed depressible and/or deformable portion is, in one embodiment, generally round in shape but the shape may be any general shape as the shape does not affect the function. In a preferred embodiment, the recessed depressible and/or deformable portion is indented slightly on the proximal end of device when it is not in use. This portion identities the area of the bulb where a thumb of a user is placed when using the device. The slight indentation of this area in a preferred embodiment provides certain advantages beyond identifying the general location for thumb placement when using the device.

In accordance with a preferred embodiment, the bulb is made from a rubber material of an elasticity which can be gently pressed and released without jarring or clicking, thereby permitting sustained control over the suction and release of the bulb. The elasticity of the recessed depressible and/or deformable portion may be the same or different from the rest of the bulb. Different materials, comprising different elasticities, may enhance user comfort and reduce hand tiredness when the device is in use. In one preferred embodiment, the recessed depressible and/or deformable portion is more elastic than the rest of the bulb identifying the place for optimal thumb placement. However, other embodiments are contemplated in which the device is uniformly made of the same material and thumb placement maybe be identified by the markings or labeling of the device. It is also appreciated that no markings are required to use the device effectively. The firmer bulb could anchor the hand while the flexible recessed depressible and/or deformable portion allows the thumb to control the bulb aspiration.

In a preferred embodiment, a recessed depressible and/or deformable portion (28) enhances control of the aspiration process when the device is used. It is preferred that the bulb is made from a rubber which is sufficiently flexible to permit control over the aspiration process and it may be all the same material or made of more than one material. Initially the device may be used to explore the oral cavity for the tonsils directly; before any aspiration occurs.

The bulb may also be manufactured using rubber which does not contain natural rubber latex

Although a round bulb would work when used in the present invention, it is not necessarily preferred as the user may have reduced control of the movement of air in and out of the bulb as the presence of a recessed depressible and/or deformable portion provides in a shape which includes such portion. Various bulb shapes including an egg shape, mushroom shape, oval shape or a round shape may be used in the present invention. Shape is independent of the presence or absence of a separate bulb portion which is depressible and/or deformable.

In one embodiment, the device is constructed from three parts, namely a bulb portion, a shaft portion, and a tip portion. The shaft portion in a preferred embodiment is a cannula. The shaft portion attaches to the bulb portion using suitable means of attachment as would be known and understood by a person skilled in the art. In one embodiment the shaft and bulb are attached by attachment means known in the art. In accordance with an aspect, the attachment means comprises a snap-fit means attaching the shaft to the bulb. In accordance with another embodiment the shaft and bulb are manufactured as a single unitary piece permanently joined together and not detachable.

In a preferred embodiment, the bulb, as described herein, is made from an elastomeric rubber, the shaft is made from a rigid plastic, and the tip portion is made from a soft flexible elastomeric material which in a preferred embodiment is transparent. At one end of the shaft portion, the bulb is attached. The tip portion is attached to the opposite end of the shaft. That is the shaft portion is located between the tip and the bulb and, in use, all 3 parts are connected and configured to establish an open airway extending from the bulb, through the shaft, and through the tip so when suction is applied and released by squeezing and releasing the bulb portion, the device will manually aspirate oral debris through the tip into the shaft. Transparency or translucency helps see the collected debris.

The tip portion is preferably soft and flexible and in a preferred embodiment, the tip portion is made of a silicone material. In another embodiment, the tip and shaft portions are made of a silicone material. In yet another embodiment, the tip, and shaft, and bulb portions are made of a silicone material. In yet another embodiment, the entire device is made of a silicone material.

The tip portion comprises an opening such as a hole at the most distil end (the end opposite the bulb) through which oral debris stones such as tonsil stones, can be aspirated. The tip opening can vary in size and the range of diameters of the opening suitable are from 1 mm in diameter to 5 mm in diameter. An even more preferred range for the opening hole at the distal end of the tip portion is a diameter ranging from about 2 mm to about 4 mm. In FIGS. 14A, 14B and 14C three aspects or embodiments are illustrated with respective openings 2 mm (18), 3 mm (18′) and 4 mm (18″). Different size openings may be possible on interchangeable tips. It is appreciated that if different tips are used interchangeably the device is best cleaned or autoclaved between uses and in some instances this is only best achieved by autoclaving the device as a whole, if it is a single piece or the parts of the device, if the device comprises parts which are assembled to create the device.

The tip portion of the device may be shaped to accommodate function. Assessment of the oral cavity by providing and exploring is done with the tip portion of the device. The tip portion may be shaped differently to be narrower or wider depending on the function of the tip and the type of use for the device whether for extraction or exploration.

Tip material is important for the device functioning in a safe way. Many current tools used by individuals who are not under medical care are unsafe and unsanitary including, amongst other things, bobby pins, Q-tips, curettage sticks, toothpicks, water piks, which incidentally do not work and also the force of the water can damage the tissue of the mucosa and cause inflammation to the tonsils. An unsanitary device clearly can introduce further bacteria into the oral cavity which could exacerbate the accommodation of the oral debris and formation of stones such as tonsil stones.

In a preferred embodiment, the device is manufactured as a single piece which is most preferably sterile when packaged for sale and preferably sold as a sterile device for a single use. Preferably the device is sold for single use followed by disposal for maximum sanitary conditions. Multiple devices could be packaged together for further cost advantages.

When manufactured as a single device, different variants may be made with different tip opening sizes or different tips could be provided. The user would purchase the version with their preferred size of tip opening depending on the size of the patient, the intended use, and the overall comfort of the patient.

Small pieces of oral debris such as a tonsil stone may be removed even with a tip opening of a larger size than the stone itself. Also, for larger tonsil stones and larger pieces of oral debris a smaller tip opening is also operable and it is possible a stone may break apart if it is larger than the opening in the tip. It is recognized that the diameter of the tip may remain unchanged by the opening hole within the tip which may have different diameters. For example, stones can break apart for aspiration or adhere to the distal tip portion for removal. For some patients it is still more comfortable to use a smaller opening rather than a larger opening particularly if the size of the opening also impacts on the full diameter of the distal portion of the tip as well. As the diameter of the hole increases, the diameter of the distal tip portion may increase to accommodate the wider opening. When the tip is inserted into the tonsil or other tissue to explore within the tissue or to reach deeper than a smaller stone a smaller tip size may be more comfortable or even necessary for the patient. The tonsil stone or oral debris need not fit inside the hole in order to be effectively removed if it can be removed in parts. If the debris is smaller than the opening of the hole then the oral debris will simply move into the device completely and this facilitates removal.

Removal of oral debris using the device may also be facilitated by blowing air into the tonsillar crypts to dislodge the debris. The expelled air generated by squeezing the bulb portion or depressing the recessed depressible portion while the tip of the device is in the crypts may push out oral debris or tonsil stones from the crypts. Furthermore, subsequent release of the bulb portion or the recessed depressible portion generates suction that then aspirates the dislodged oral debris.

Removal of oral debris using the device may also be facilitated by plucking or scooping the oral debris out of the crypts.

Preferred suitable material for the tip portion is a soft elastorneric, transparent or translucent material, including but not limited to silicone. One example but certainly not the only option is a brand known as Dynaflex™. Thermoplastic elastomers (TPE) would also be another specific example. SR549M polypro plastic would also be yet another specific example. Persons skilled in the art would know other suitable materials. Transparency at the tip portion is preferred so the stones are more clearly visible. However, even an opaque tip would work but is less preferred.

In another embodiment, suitable material(s) for the tip and shaft portions is a soft elastomeric, transparent or translucent material, including but not limited to silicone. One example but certainly not the only option is a brand known as Dynaflex™. Thermoplastic elastomers (TPE) would also be another specific example. SR549M polypro plastic would also be yet another specific example. Persons skilled in the art would know other suitable materials. Transparency at the tip and/or shaft portion(s) is/are preferred so the stones are more clearly visible. However, even an opaque tip and/or shaft would work but is less preferred.

In another embodiment, suitable material(s) for the tip, shaft, and bulb portions is a soft elastomeric, transparent or translucent material, including but not limited to silicone. One example but certainly not the only option is a brand known as Dynaflex™. Thermoplastic elastomers (TPE) would also be another specific example. SR549M polypro plastic would also be yet another specific example. Persons skilled in the art would know other suitable materials. Transparency at the tip, shaft, and/or bulb portion(s) is/are preferred so the stones are more clearly visible. However, even an opaque tip, shaft, and/or bulb would work but is less preferred.

In another embodiment, suitable material(s) for the entire device is a soft elastomeric, transparent or translucent material, including but not limited to silicone. One example but certainly not the only option is a brand known as Dynaflex™. Thermoplastic elastomers (TPE) would also be another specific example. SR549M polypro plastic would also be yet another specific example. Persons skilled in the art would know other suitable materials. Transparency of the device is preferred so the stones are more clearly visible. However, even an opaque device would work but is less preferred.

In hospital settings where autoclaves are available, then a preferred material of manufacture would be plastics, rubbers, and elastomers which are autoclavable. In this instance, the device could be safely re-used after it has been autoclaved.

Turning to the Figures, FIGS. 1 to 13 illustrate a preferred embodiment of the oral debris extractor device 10 comprising a bulb suction means 20. A preferred bulb suction means is illustrated in FIG. 41. In FIGS. 1 to 10 the bulb suction means comprises, in this particular embodiment, a squat or truncated pear-shaped bulb 20. The bulb 20, in this particular preferred embodiment, has a generally pear-shaped (pyriform) overall shape. The thumb positioning region 12 at the base of the bulb (defined as the proximal end) marks where a recessed depressible portion (the recessed portion) is present on the base of the bulb 20. The recessed portion 28 is illustrated, for example, in FIG. 8 and in the cross section illustrated in FIG. 10, for this particular embodiment.

Further details of the preferred embodiment are illustrated in FIGS. 1 to 13. In a preferred embodiment, grip means exemplified as circumferential ridges 8 are present on the surface of bulb 20 and in this preferred embodiment the ridges 8 circumferentially surround the bulb 20 in parallel lines which are spaced apart. Spacing between circumferential ridges 8 need not be an even spacing. Also the ridges need not fully circumscribe the surface of the bulb. In other embodiments one skilled in the art would appreciate that a variety of patterns of surface ridges or other shapes would work in the present invention. In an enlarged cross-section, illustrated in FIG. 43, ridges 8 are more clearly visible on bulb 20. In a preferred embodiment, the ridges are preferable of a height from about 0.1 mm to about 10 mm. In another preferred embodiment from about 0.25 mm to about 2 mm and in another preferred embodiment from about 0.5 mm to about limn. It is appreciated that the width of the ridges can also vary and in the illustrated embodiment would preferably range from, about 0.5 mm to about 10 mm.

The ridges 8 are generally raised from the surface of the bulb 20. The function of the ridges is to assist with gripping or holding bulb 20 as well as identifying the placement of one or more digits on a hand and, in some instances, for users who have lost feeling or sensation in a portion, particularly the tips, of their digits. The ridges 8 are one example of grip means for when the user is holding the bulb 20. These assist in reducing the likelihood the bulb will slip when held and when in use. Persons skilled in the art would appreciate that the pattern of the ridges is only one possible pattern. Other patterns are possible. The ridges need not all completely circumscribe the surface of bulb 20. Incompletely circumferential ridges could work but possibly not as well depending on the pattern of the ridges. One skilled in the art would also appreciate that grooves could replace the ridges and also create a different form of grip means on bulb 20 (not illustrated).

In a particularly preferred embodiment the ridges 8 are raised above the surface of the bulb 20 and the height of ridges 8 above the surface of the bulb 20 is preferred to be in the range of about 1 mm to about 5 mm in height. In the illustrated preferred embodiment shown in FIGS. 1 to 10 the ridges are a uniform height. It is submitted that a uniform height of the ridges 8 may be more comfortable for the user and provide greater stability when gripping the device 10 particularly when in use but it is not required that the height be precisely uniform. A person skilled in the art would appreciate that if too high the ridges 8 may be uncomfortable or create instability to the user when the bulb 10 is held; however, in an embodiment for use by users with loss of feeling in their finger tips the ridges or other grip means must be felt by the user. For this reason ridges are preferred over grooves' however, the shape and configuration of the raised portions could vary as would be appreciated by one skilled in the aft. The ridges 8 optimally provide additional grip and stability of the bulb 10 for the user without discomfort or instability.

It is advantageous if the bulb 10 is stable in the user's hand. Ridges 8 provide one means for increasing the stability of the bulb when in use. The shape of the bulb 20, in this preferred embodiment, also facilitates ease of holding the bulb 20 when in use in order to maneuver the bulb when it is in an oral cavity. The device 10 may function as both an exploratory device as well as an extraction device. A bulb 20 with a shape which facilitates maneuverability while maintaining stability in the hand of the user (so it does not slip in use) is preferred.

One preferred shape is illustrated in FIGS. 1 to 13. This shape is substantially a truncated pear shape also known as a pyriform shape. The direction of the bulb is suggestive of an inverted cone, when in use and this is also known as obconic. Overall, in use, the bulb 20 is considered to have an obpyriform shape which suggests the bulb is an inverted pear shape. The direction of the pear shape is obconic (inverted) when the device 10 is in use. One may also refer to the shape as mushroom-shaped.

The recessed portion facilitates holding the device when there is reduced hand dexterity providing an advantage for a steadier hand.

In, for example, a patient with Multiple Sclerosis who is still ambulatory, they may be able to use this device on their own providing them with greater independence in their own self care because of the various elements of the device which together provide a device which is not only more securely gripped but easier to feel for placement of the thumb and fingers on the bulb. The hand does not have to change position to operate the device for squeezing and releasing the bulb.

While the shape illustrated in FIGS. 1 to 13 is a preferred embodiment shape, other suitable shapes would be appreciated by persons skilled in the art including, without limitation, ovoid shapes, obovoid, clavinate and obclavinate, turbinate, as examples and all of which may appear truncated at the proximal end if a recessed portion is located in that position.

For ease of reference, the bulb end 15 refers to the end of the device 10 which is held in the hand of the user when the device is in use. The tip end 17 refers to the end of the device 10 which goes into the mouth (oral cavity). At the bulb end 15 of device 10 the bulb 20 has a recessed portion 28 which in the illustrated preferred embodiment is circular; however, one skilled in the art would understand that other shapes for the recessed portion 28 would work. For example, the recessed portion need not be perfectly circular.

The bulb 20 is preferably made of rubber, either natural or synthetic, material which holds its shape when not in use but can be deformed with ease when in use but returns to its shape as air returns into the bulb. The recessed portion 28 comprises an equivalent deformability or even greater deformability than the rest of the bulb. In a preferred embodiment, the recessed depressible and/or deformable portion may be made of the same rubber material as the bulb or it could be, amongst other things, made from a different material to further facilitate comfort and control. It would be appreciated by a person skilled in the art that the recessed portion could be made from a rubber or other elastomeric polymer which is more or less deformable than the material of the rest of the bulb.

The preferred shape as illustrated in FIGS. 1 to 13 permits a user to hold the bulb 20 by placing a thumb 52 in the recessed portion 28 and placing an index finger 54 on the opposite side of the bulb 20 to provide a placement of the hand which enhances stability by reduces slippage when in use. FIG. 11 illustrates the placement of the thumb on the recessed portion 28 without pressing the thumb 52 into the bulb 20. FIG. 12 illustrates when the thumb 52 and index finger 54 squeeze together and the thumb presses into the recessed portion as air is squeezed out of the bulb 20.

In one embodiment, a tip portion 16 may be separate from a shaft portion 14 and a demarcation line 13 may be visible, demarking the tip 16 from the shaft 14.

In the preferred embodiment a recessed portion 28 is provided at the bulb end 15 of the bulb 20. Another embodiment is also possible by providing separate materials which may vary in elastomeric properties such that the recessed portion 28 comprises a greater or lesser degree of elasticity than the rest of the bulb.

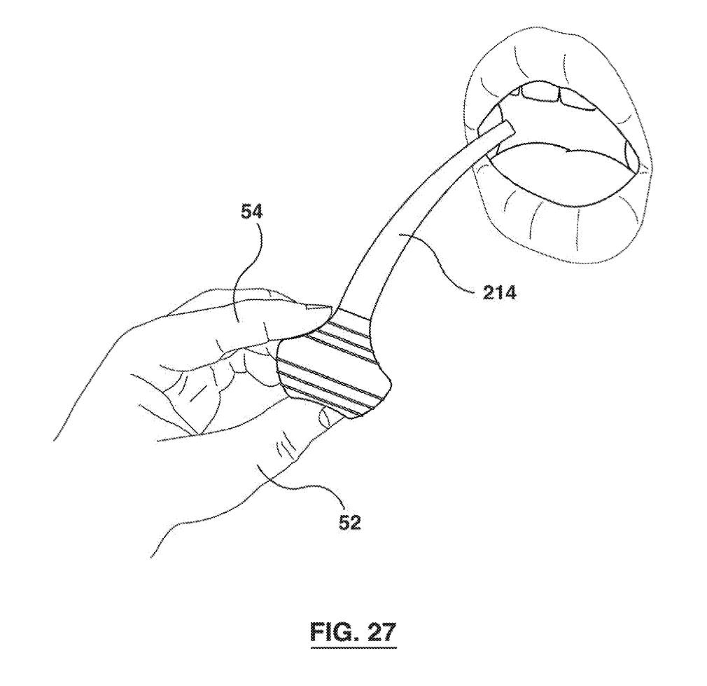

In another preferred embodiment as illustrated in FIGS. 15 to 27, the curved shaft 214 is a single continuous piece extending from the distal end of mushroom-shaped bulb 220 to the tip region 216.

In another embodiment, the bulb 120 may not comprise a recessed portion as illustrated and therefore is less preferred, for example, in FIG. 61. In FIG. 61, a less preferred oral debris extractor device 110 is illustrated wherein the bulb portion 120 is rounded and does not comprise a recessed portion 28. The placement of the thumb 52 may be on the side of bulb 120 as illustrated in FIG. 61.

FIG. 61 illustrates a less preferred embodiment of the present invention but it is still a possible embodiment. The embodiment illustrated in FIG. 61 is less preferred because, amongst other things, the straight shaft 114 is straight without a preferred curve. An example of a piece of oral debris or tonsil stone or oral debris 98 present in the straight shaft 114 after aspiration is illustrated in FIG. 61. The tonsil stone or oral debris 98 is visible in the straight shaft 114 because the straight shaft 114 is illustrated in its preferred material which is translucent or transparent so the oral debris is visible after collected by device 110.

In the preferred embodiment illustrated in FIGS. 1 to 13 shaft 14 extending from the distal end of bulb 20 is curved as opposed to straight. A straight shaft is illustrated in FIG. 60.

In a preferred embodiment shaft 14 is made from a rigid plastic which preferably is either transparent or translucent so extracted oral debris is readily seen when collected. The preferred curved shape of shaft 14 facilitates placement of device 10 in the mouth from the side of the mouth which reduces the likelihood of a gag reflex caused by touching that part of the back of the throat which most frequently causes a gag reflex. The curved shaft in combination with the elastomeric tip aid in alleviating and/or reducing the frequency of incidents of gag reflex. In a preferred embodiment the shaft is generally tubular with a centrally located opening which extends along the length of the shaft connecting a bulb suction means to a tip portion. Other shapes of the shaft are possible, other than tubular. An example of another shape is illustrated in FIG. 59.

It would be appreciated by a person skilled in the art that the length of the shaft, the shape of the shaft, and the tip length (and tip opening size) may vary. Size variations may be warranted depending on the size of the head and mouth of the patient receiving treatment with the device. For example larger mammals with tonsils may require a proportionally larger tip and shaft for use. While bulb size may also change, bulb size preference is also related to the hand size and comfort of the user as well. Various bulb could use in the present invention. Some examples of different bulb shapes are found, for example in FIGS. 57 and 58. It is noted that the device may have benefit in mammals other than humans. Adjustments in the length of the shaft and the tip can facilitate the use in other mammals including larger animals such as horses.

It is known that tonsil stones develop in the tonsillar crypts, most often in the palatine tonsils but also in lingual tonsils. Palatine tonsils are the ones that humans have at the back of their throats and the palatine tonsils are partially visible when the mouth is opened. In addition to humans, other animals have palatine tonsils, including, but not limited to, the following: other primates, cattle, sheep, goats, and elephants. Horses do not have palatine tonsils but instead have small aggregates of lymphoid tissue, similar to tonsillar tissue, located throughout their throat. It is possible that the device could be used, for example, at the same time as another surgery. In dogs, for example, often the teeth are cleaned at the same time as another surgery and/or the pet is placed under anesthetic for cleaning of the teeth. At the same time any tonsil stones or other oral debris could be removed using an oral debris extractor in accordance with the present invention, as a veterinary medical device.

In another variation of the present invention the tip is a separate piece which is mounted on and/or attached to the shaft and may be ejected through the use of an ejector button. An example of one embodiment comprising an ejector is illustrated in FIGS. 29 to 36. An optional additional piece comprises an ejector body 30 joining the shaft 14 to the bulb 20. One embodiment of an ejector body 30 is illustrated in FIG. 42. In another possible embodiment the ejector body portion 30 is an integral part of shaft 14. In another embodiment a distal portion of shaft 14 is ejected along with tip 16. A further embodiment is illustrated in FIGS. 45 and 46. In the embodiment illustrated in FIGS. 45 and 46 the ejector body 30 is an integral portion of shaft 14. Shaft 14 is connected to bulb 20′ by a snap-fit connection means 56. The snap-fit connection means 56 is generally comprised of two parts. The shaft 14 comprises a male part 58 which snap fits into a female part 60 located at the distal end of bulb 20. In the embodiment illustrated in FIGS. 45 and 46 the ejector button 34 is connected to an ejector ring 36 which when pressed pushes the tip portion 16 off of shaft 14 for easy disposal without the user having to touch the tip. The tip could readily be ejected into a sanitary container to collect the tips for later autoclaving or sanitary disposal.

FIGS. 47 to 56 illustrate another embodiment with a variant tip portion 16′ which optionally attaches to shaft 14 or in another embodiment could be integral with shaft 14. In accordance with another aspect, tip portion 16′ may optionally attach to shaft 14 by pushing over top of the distal end of shaft 14 or in another embodiment screwing on to the distal end of shaft 14.

FIGS. 37 to 40 illustrate yet further embodiments. According to one aspect the embodiments illustrated in FIGS. 37 to 40 comprise an ejector body 30 and ejector button 34. In accordance with another aspect of the invention the shaft 14 is not uniformly curved along its length. Rather at the joint between shaft 14 and tip 16 there is a bend region 22.

FIGS. 57 to 60 illustrate yet further embodiments. As illustrated in FIG. 59 the shaft portion 14″ is a different shape and the relative lengths between shaft 14″ and tip 16″ illustrate a shorter shaft 14 and a longer tip 16. In accordance with another embodiment bulb 20′ is more ovoid in overall shape. At the bulb end 15 of bulb 20′ the generally ovoid shape is truncated at the location of recessed portion 28 illustrated as centrally located at the proximal end of bulb 20′.

An example of the connection between shaft (14) and bulb (20) is shown in cross-section in FIG. 10. This embodiment of the connection is shown for illustrative purpose only, and should not be interpreted to be limiting on the types of connections possible for the present invention. This embodiment of the connection also does not reflect the manufacturing process of the device (10) or its components. A person of skill in the art will appreciate that different types of connections are possible and that different manufacturing processes can be used to produce the desired connection. The same applies to connections between ejector body (30) and bulb (20), an example of which is illustrated in FIG. 43.

Turning to FIG. 44, if the ejector body (30), or other components of the device, is to be manufactured by injection molding, the interior channel (32) of the ejector body (30) can be designed as a straight channel in order to facilitate the injection molding manufacturing process. Similarly, if injection molding is to be used for the manufacture of embodiments illustrated in FIGS. 1-10, FIGS. 15-24, FIGS. 47-56, and FIGS. 57-60, a person of skill in the art is able to redesign the shaft (14) and/or bulb (20) to accommodate the injection molding manufacturing process. Other manufacturing processes may be used and a person of skill in the art is able to redesign the components as needed to accommodate the corresponding manufacturing processes.

In use, the oral debris extractor may be used to explore the oral cavity to determine the extent of oral debris present and the nature of the oral debris. When oral debris is located the extractor may be used to remove the debris. Prior to insertion of the extractor into the oral cavity, an initial visual assessment of the oral cavity is conducted. If the patient or individual is using the extractor on their own then they would use a mirror to look in their own mouth. If the patient or individual has the assistance of a care provider then the care provider would look inside the oral cavity if they are the person who will be using the extractor to remove any oral debris.

The determination of whether a caregiver uses the device or the person on their own uses the device is based not only on personal choice but also on ability to manage the device depending on the nature of the patient's condition(s). For example, motor neuron diseases with injury at the base of the skull affects the control and dexterity of the hand. Accordingly, depending on the extent of the patient's ability to use their hands and/or control their head a decision would be made whether a caregiver would be best to use the device on the patient or individual. The determination is a practical one based on physical limitations. Even if there are no restricting physical limitations, it may still be a personal choice to use a caregiver to provide the assistance. An advantage of the present device is that it can be used interchangeably by either a caregiver or the individual without modification. A caregiver may be a layperson who is not, and has not been, involved with the healthcare industry.

Assessment of the nature and type of oral debris would be made by the user and a determination would be made as to the type of extractor which is best for that type of debris. For example, a decision may be made to select a particular size of tip opening as the preferred choice. One of skill in the art would know how to make the assessment. In order to assess the debris, the patient or individual may also be required to open their mouth wide and stick out their tongue. This action widens the jaw, allowing the patient or caregiver to see inside the cavity, including both the tonsils and the surrounding tissues.

When visually assessing the oral cavity, in order to see the tonsils and look for the presence of tonsil stones, it is best to have the tongue extended. A tonsil stone may be identifiable, for example, as a white mass visible on or in the tonsil tissue. Once it is seen, the device of the present invention can be used to remove it and you can confirm its removal by looking again and noticing that the white mass is no longer present. The patient may also be able to confirm that they can no longer feel the stone but it is appreciated that not all patients feel the stone. Similarly not all patients may be able to speak and this is of course particularly true in a veterinary setting.

If there are very large food particles then, initially, a Swipe with the device may be warranted before further Use of the oral extractor device. The curve in the device acts as a finger and can be used as a substitute for a finger swipe since the device may be made with a thermoplastic elastomer (TPE) which is a material that both the U.S. Food & Drug Administration (FDA) and Health Canada have approved for use in humans and the bent shape emulates a finger.

When looking in the oral cavity the user would visually assess what they can see in terms of oral debris. For example, the extractor is particularly useful for the removal of tonsil stones and in this instance the user of the device would locate what tonsil stones they can see by eye. Completing this assessment visually first helps the user to know where to direct the tip of the device when it is in the mouth.

Once the user locates the position and nature of the tonsil stones or oral debris the user holds the bulb of the device in their hand of preference. In a preferred embodiment the user's thumb is placed on the recessed portion or designated thumb placement area of the bulb.

Then the bottom of the bulb, where the thumb recessed portion is located, is depressed by the user. In a preferred embodiment the user fully depresses the bottom of the bulb using the thumb until it is fully depressed. Ensuring that the thumb remains in the recessed indentation the user establishes their grip on the remainder of the bulb. In a preferred embodiment, the index and middle fingers securely cradle the bulb of the preferred shape illustrated in FIGS. 11 to 13. The index and middle fingers are used to securely cradle the bulb gripping a plurality of raised ridges which comprised a gripping means. This step is completed while the device is still outside of the oral cavity.

Keeping the thumb in its position depressing the recessed indentation portion on the bottom of the bulb, the user places the tip of the device into the oral cavity and directs the tip to the location of the observed oral debris or tonsil stone(s). Once the tip of the device is placed in proximity to the oral debris or stone, the user releases the thumb, very slowly and gently, thereby creating suction action and the debris will be aspirated into the hollow shaft of the device through the opening in the tip. Once the bulb has returned to its original state it is removed from the oral cavity.

One of skill in the art will appreciate that, depending on the nature of the oral debris or stone, all of the debris may not enter the hollow shaft at once. It will also be appreciated by one with skill in the art that the oral debris or stone may not enter the hollow shaft and instead may remain adhered to the tip of the device by the suction. Either way the debris is now contained by, or connected to, the oral debris extractor and when the extractor is removed from the oral cavity, the debris will also be removed at the same time.

It is understood and appreciated that remaining debris will require further uses of the extractor until all debris is sufficiently removed from the oral cavity.

When the device has been removed from the oral cavity the debris should be readily visible either on the tip of the device or as seen through the translucent or transparent tip and shaft material. If no visible debris is apparent, it is possible that no debris was collected and the steps for collection axe then repeated until debris is visible at the tip or in the shaft of the device.

Once the debris has been aspirated (into the device) or plucked (using the tip of the device) the device can be cleared of any debris by depressing and releasing the bulb repeatedly while immersing the tip under running water. Continue repeating the depressing and releasing of the bulb until there is no debris visible at the tip or in the hollow shaft of the device.

These steps may be repeated to ensure that all tissues and locations of concern within the oral cavity are free of debris without need to change the extractor during the repetition of the steps and once all debris has been collected and removed then the device, if disposable, would be appropriately thrown away. If the device was of the type for reuse then it would be sent for autoclaving or sterilization of the device. Disposable tips would always be thrown away and not reused. This marks the completion of the process. The frequency of the process would depend on the condition(s) of the individual or patient, the nature of the overall health of the patient and, accordingly, would be determined based on need. As the device is inexpensive and easy to control and manage then it is possible to always have a fresh, sanitary device at hand when needed.

In accordance with another embodiment, the device may be used to expel air into the pits or crypts of a tonsil or other tissue or part of the oral cavity in which tonsil stones or oral debris deposits are located either by feel or observed visually. In some instances the debris may only be partially visible or, in the case of a tonsil stone, it may be deep in a crypt or pit and its presence is only known by the feel and not observable by the eye.

In the embodiment for expelling air into a specific location in the oral cavity the preferred method is different from the aspiration method. In this case the user's thumb is placed in the recessed portion located on the bottom of the bulb and the index and middle fingers are used to securely cradle around the raised ridges present on the surface of the bulb. It is appreciated that the ridges may be in any configuration. In this embodiment, for expelling, the user does not depress the bulb before placing the device into the oral cavity. The tip of the device, comprising an opening hole, is placed into the oral cavity and then into the crypt or crevice in which the debris is believed to be located.

At this time the bulb is depressed by the thumb causing air to be expelled into the cavity, crypt or crevice. The expulsion of the air, when it comes in contact with the debris, will facilitate the debris removal by dislodging the debris and render it easier to then remove. An advantage of the device is that the step of expelling the debris can be immediately followed up by removal while the device is still in the oral cavity as the bulb has been depressed and upon release the debris will either be aspirated or plucked by the device. Alternatively, the user may choose to swipe with the device (curved shaft and tip) or simply spit out the debris which has been dislodged by this method.

In another embodiment of the method, the bulb may be removed from the oral cavity and then the steps, set out above, for aspiration can be followed. To aspirate, simply depress the bulb following the previous steps when the device is outside the mouth, preferably, and then insert the tip of the device into the oral cavity as explained in the prior steps, in the vicinity or proximity of the debris, and aspirate the debris into the hole in the tip to be collected in the hollow shaft to which the tip is joined or otherwise integrally molded.

Disposal of the debris from the device is the same as outlined above once the oral debris has been removed from the oral cavity. Repeat if necessary.

The tip portion of the device may also be used, in another embodiment, as an explorer of the oral cavity and also to dislodge the debris from its location in the oral cavity. Rather than using air, the tip which is made from a gentle and safe material which will not damage the oral mucosa, is suitable for dislodging and/or plucking, or pushing or pressing, safely by physical action using the tip of the device located at the end of the curved shaft the debris from its location in the oral cavity after which it can be removed by spitting, swiping with the device, or using the device as an aspirator.

In accordance with another embodiment an additional step may be used when introducing the tip of the device into the oral cavity to assist in reducing any gag reflex. The user may first choose to gently touch the tip against the inside cheek of the user first on one side and then on the other side before reaching deeper into the oral cavity. This allows the patient to adjust to the presence of the device in the oral cavity and helps to reduce the natural gag reflex. This is greatly facilitated by the curvature present in the shaft portion of the device.

The curvature of the shaft also provides a preferred ergonomic design for the use of the device. This feature is one aspect of the preferred embodiment which provides benefits over a straight shaft. Although the use of the bulb of the present invention on a straight shaft is possible, it is not the most preferred embodiment. With the curvature additional benefits are recognized including easier accessibility into crypts, folds, and pits, great patient comfort, and assistance in exploration of the oral cavity in a safer way. It is noted that most stones hide in crypts\folds found on either side of the patients oral cavity.

In accordance with an embodiment of the present invention, there is provided an oral debris extractor for use to remove oral debris from an oral cavity of a mammal suffering from dysphagia, a neurologic disease, a motor neuron disease, diabetes, ALS, Multiple Sclerosis and other diseases which underlie deterioration of a person or mammal's muscular or neuronal well being. Conditions which, for example, reduce feeling on the tips of digits such as fingers and the thumb make it difficult for users to hold a smooth or completely round bulb. While bulbs with a thumb region will be operable and the thumb region may be defined by a recessed portion which or it may be defined simply by a material of greater elasticity than the rest of the bulb or it may be both of these features. The thumb region may be defined by a separate material. An advantage of this embodiment of the present invention is that the portion of the bulb which is distal from where the bulb connects with the shaft, provides an identifiable region by either material or recessed shape, or both, whereby a person with reduced sensitivity in their fingertips because of an underlying illness, disease or condition, would still be able to readily recognize where the thumb is optimally placed in order to use the device on their own. Similarly a grip means on the surface of the bulb, which in the illustrated preferred embodiments comprises raised lines on the surface of the bulb but other types of raised grip means would be appreciated by a person skilled in the art, provide something to actually feel when holding the bulb in use. The user with reduced feeling and sensation in their finger tips and thumb is able to stably and effectively still use the bulb because they are able to feel the location of the digits and thumb on the surface of the bulb when they are holding it. Accordingly, in the illustrated preferred embodiment parallel ridges are used around the surface of the bulb other shapes would be possible. The ridges could be wider and discontinuous in possibly other shapes (squares, circles, triangles, rectangles and all other shapes) are conceivable as operable in the present invention. In a preferred embodiment the pattern is defined so that it is recognized by a user who has reduced sensitivity, feeling or sensation.

In the present invention, the preferred shape of the bulb is a mushroom shape such that the user's hand is stable and able to control the device when in use as an oral debris extractor, explorer or assessment tool. The user is able to deftly depress the recessed region under the thumb even if they lack hand dexterity because of limited feeling in the tips of the fingers or thumb area. Similarly the placement of the remaining digits below the cap portion of the mushroom shaped bulb is facilitated by raised ridges which the user is able to feel. Accordingly the ridges must protrude sufficiently above the surface of the bulb to be felt by the user but not too high that stability for holding is lost by the awkwardness of the grips. Although not illustrated one skilled in the art would appreciate that locations for the remaining digits may be identified on the bulb as well by particular patterns of ridges, for example.

When in use the fingers which are positioned around the base of the bulb and rest closer to where the base of the bulb portion meets the shaft portion provide a foundation against which the thumb is able to depress the recessed portion or the portion made of a different material to expel the air from the device.

The present device may be used for the extraction, expulsion, assessment, and aspiration of oral debris in an oral cavity in self care or assisted care when the patient or individual has any of a number of conditions that compromise motor or neuron capacity of the individual including, but not limited to, Multiple Sclerosis, brain injury, spinal cord injury, ALS, diabetes, stroke, any of a number of cancers, neurological conditions, halitosis, dysphasia, ear pain, throat pain, and tonsil enlargement.

Other and further embodiments of the present invention would be understood to a person skilled in the art in the context of the spirit of the invention as determined when the specification is read as a whole including without limitation the following claims.

All references, patents, and patent applications cited; hyperlinked, and/or specifically identified herein are hereby incorporated by reference in their entirety.

1.-103. (canceled)

104. A device for removing oral debris from an oral cavity of a mammal, the device comprising:
a bulb of resilient material, the bulb defining an interior air space;
a curved shaft comprising a first shaft end and a second shaft, the curved shaft further comprising a channel, the channel extending through the length of the curved shaft from the first shaft end to the second shaft end;
the first shaft end connected to the bulb such that the channel is contiguous with the interior air space in the bulb when the first shaft end is connected to the bulb;
the bulb comprising a first bulb end connected to the first end of the curved shaft and a second bulb end substantially opposite the first end, the second bulb end comprising a recessed portion to receive a user's thumb;
the second shaft end comprises a tip portion comprising a diameter slightly narrower than the diameter of the second shaft end;
the channel extending contiguously through the tip.

105. The device according to claim 104, wherein the bulb further comprises grip means on an outer surface of the bulb.

106. The device according to claim 105 wherein the grip means comprise at least one raised ridge extending partially or fully circumferentially on the outer surface of the bulb.

107. The device according to claim 106 wherein the grip means further comprises a plurality of substantially circumferential ridges parallel lined along the outer surface of the bulb.

108. The device according to claim 107, wherein the ridges are raised above the outer surface of the bulb at a height from about 0.1 mm to about 10 mm in height.

109. The device according to claim 108, wherein the width of each ridge is from about 0.25 mm to about 2 mm.

110. The device according to claim 109, wherein the width of each ridge is 1 mm.

111. The device according to claim 110 wherein the recessed portion is adapted to receive a user's thumb.

112. The device according to claim 111 wherein the recessed portion comprises an indentation.

113. The device according to claim 112 wherein the recessed portion comprises a material of greater elasticity than the rest of the bulb.

114. The device according to claim 113, wherein the bulb comprises a shape selected from the group of shapes consisting of obconical shapes, ohpyriform shapes, pyriform shapes, turbinate shapes, obclavinate shapes, pear-shaped shapes, and mushroom-shaped shapes.

115. The device according to claim 114, wherein the bulb is made from a natural or a synthetic rubber.

116. The device according to claim 114, wherein the bulb is made from silicone.

117. The device according to claim 116, wherein the shaft is translucent or transparent.

118. The device according to claim 117, wherein the tip, the shaft, and/or the bulb are made from silicone individually or in combination.

119. The device according to claim 118, wherein the entire device is made from silicone.

120. The device according to claim 119, wherein the device is a unitary piece.

121. A method of using the oral debris device according to claim 104, the method comprising the steps of inserting the device into a patient's oral cavity, exploring a first side of the oral cavity, exploring a second side of the oral cavity, exploring a back of the oral cavity, or exploring crypts in the oral cavity to detect the presence of oral debris.

122. A method of aspirating oral debris from an oral cavity using the device of claim 104, the method comprising the steps of:
a) placing a user's thumb in the recessed portion;
Ii) placing the user's index finger and middle finger around the bulb while the thumb is placed in the recessed portion;
c) with the device outside of the oral cavity depress the recessed portion using the thumb and squeeze the bulb with the fingers releasing air out of the bulb;
d) place the tip and a portion of the shaft of the device into the oral cavity of a mammal extending into the cavity only the distance required to reach the oral debris or the area to be explored for oral debris,
e) place the tip of the device in close proximity to the oral debris; and
f) slowly release the thumb from the recessed portion to suction the debris into the device;
g) gradually cease squeezing the bulb with the fingers and once the debris has entered the device remove the device from the oral cavity.

123. The method according to claim 122, wherein the recessed portion is fully depressed using the thumb in step c) and the slow release in step f) is stopped during the release to suction further debris into the device.

124. A method of expelling lodged oral debris from a location in an oral cavity using the device of claim 104, comprising the steps of:
a) placing a user's thumb in the recessed portion;
h) placing user's index and middle fingers around the bulb;
c) placing the tip of the device into the oral cavity; and
d) depressing the bulb using the thumb and expelling air to dislodge the oral debris.

125. The method of claim 124 wherein the lodged oral debris is located in an oral crypt.

126. The method according to claim 125, further comprising the step of slowly releasing the user's thumb from the bulb for aspirating dislodged oral debris.

127. A method of plucking oral debris from an oral cavity using the device of claim 104, comprising the steps of:
a) pressing the tip around an area of the oral cavity where the oral debris is located;
b) dislodging the oral debris by pressing or pushing the oral debris; and
c) removing the oral debris after it has been dislodged.

128. The method according to claim 127, wherein the area of the oral cavity is a crypt in the oral cavity.

129. The method according to claim 128, wherein step c) further comprises swiping the oral debris out of the oral cavity using the tip or the shaft.

130. The method according to claim 128, wherein step c) further comprises aspirating the oral debris using the device.

131. A bulb comprising:
a first and a second end;
means for attaching the bulb to a shaft disposed at the first end;
a recessed portion adapted to receive a thumb disposed at the second end; and
grip means disposed on an outer surface of the bulb.

132. The bulb according to claim 131, wherein the grip means is raised on the outer surface of the bulb.

133. The bulb according to claim 132, wherein the recessed portion comprises an elastomeric material, different from the material of the bulb.

134. The bulb according to claim 133, wherein the elastomeric material has a greater elasticity than the material of the bulb.
